(19) United States
(12) Patent Application Publication
(10) Pub. No.: US 2026/0363653 A1
(43) Pub. Date: Nov. 19, 2026

(54) MULTI-FUNCTIONAL ULTRASOUND SCANNER

(71) Applicant: FUJIFILM SONOSITE, INC., Bothell, WA (US)

(72) Inventors: Harald Fiedler, Bothell, WA (US); Jes Smith, Bothell, WA (US); Craig Chamberlain, Seattle, WA (US); Saeed Aliakbari, Snohomish, WA (US); Jimin Zhang, Bellevue, WA (US); Thomas Endres, Sagle, ID (US)

(21) Appl. No.: 19/212,283

(22) Filed: May 19, 2025

(51) Int. Cl. A61B 8/08; A61B 7/04; A61B 8/00; G16H 40/63

(52) U.S. Cl. CPC A61B 8/0841; A61B 7/04; A61B 8/42; A61B 8/463; A61B 8/464; A61B 8/5207; A61B 8/54; G16H 40/63; A61B 2562/0204

(57) ABSTRACT

Ultrasound systems, ultrasound scanners, and methods for using the same are disclosed. In some embodiments, an ultrasound system includes a display device configured to display a user interface for the ultrasound system, an ultrasound scanner having at least one transducer array configured to transmit ultrasound at a patient anatomy and receive reflections of the ultrasound from the patient anatomy, and an infrared sensor configured to receive infrared radiation from the patient anatomy. The ultrasound system also includes a processor system configured to: generate, based on the reflections of the ultrasound and the infrared radiation, an image of the patient anatomy and cause the user interface to display the image of the patient anatomy.

### FIELD

Embodiments disclosed herein relate to ultrasound systems. More specifically, embodiments disclosed herein are related to an ultrasound scanner that performs additional functions in addition to transmitting ultrasound at a patient anatomy and receiving reflections of the ultrasound from the patient anatomy.

### BACKGROUND

Despite decades of deployment, conventional ultrasound systems can still have limiting deficiencies, including inadequacies in needle visualization for the extremely common needle insertion procedure. In many cases, a clinician inserts a needle blindly into a patient. Infrared systems detect heat signatures using electromagnetic waves (e.g., infrared radiation) and are sometimes used instead of ultrasound systems. However, infrared imaging systems are generally limited to C-plane imaging, which can limit their utility for needle tracking.

Ultrasound systems use inaudible sound waves to generate image data; the sound waves themselves are not consumed by the operator. Rather, the operator often uses a stethoscope for consumable audio, usually asynchronously from the ultrasound image data, as simultaneous operation of the ultrasound probe and stethoscope is cumbersome. The audio data is usually not recorded or machine processed, and therefore its interpretation is subjective. Accordingly, patients may not receive the best care possible.

Many conventional ultrasound systems include guidance mechanisms to assist a clinician, like a graphic displayed on an ultrasound machine that indicates a probe placement to achieve an image view, or a ‘quality indicator’ showing feedback on how good the currently obtained image is. These mechanisms, when enabled, can usually be viewed by other personnel in the room. Many clinicians, however, do not enable the guidance mechanisms out of ego, fear etc., which is exacerbated when their use can be witnessed by the other personnel. Hence, clinicians may not utilize the ultrasound system to its fullest capabilities, and may capture sub-optimal ultrasound images.

For at least these reasons, patients may not receive the best care possible when using conventional ultrasound systems.

### SUMMARY

Ultrasound systems, ultrasound scanners, and methods for using the same are disclosed. In some embodiments, an ultrasound system includes a display device configured to display a user interface for the ultrasound system, an ultrasound scanner having at least one transducer array configured to transmit ultrasound at a patient anatomy and receive reflections of the ultrasound from the patient anatomy, and an infrared sensor configured to receive infrared radiation from the patient anatomy. The ultrasound system also includes a processor system configured to: generate, based on the reflections of the ultrasound and the infrared radiation, an image of the patient anatomy and cause the user interface to display the image of the patient anatomy.

In some other embodiments, an ultrasound system includes a display device configured to display a user interface for the ultrasound system, an ultrasound scanner having at least one transducer array configured to transmit ultrasound at a patient anatomy and receive reflections of the ultrasound from the patient anatomy, and an infrared sensor configured to receive infrared radiation from the patient anatomy. The ultrasound system also includes a processor system configured to: generate, based on the reflections of the ultrasound, an ultrasound image of the patient anatomy, generate, based on the infrared radiation, an infrared image of the patient anatomy, and cause the user interface to display the ultrasound image and the infrared image.

In yet some other embodiments, an ultrasound system includes an ultrasound scanner having at least one transducer array configured to transmit ultrasound at a patient anatomy and receive reflections of the ultrasound from the patient anatomy and at least one sensor different from the at least one transducer array and configured to generate sensor data based on radiated energy from the patient anatomy. The ultrasound system also includes a processor system configured to generate, based on the reflections of the ultrasound and the sensor data, an image of the patient anatomy.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound system in an environment with a multi-functional scanner during an ultrasound examination.

FIG. 2 is an example implementation of some embodiments of the ultrasound system from FIG. 1.

FIG. 3 illustrates some embodiments of an example ultrasound system with a multi-functional ultrasound scanner.

FIG. 4 illustrates some embodiments of an example ultrasound system for processing data from a multi-functional ultrasound scanner.

FIG. 5 illustrates some examples of an example ultrasound system for processing data from a multi-functional ultrasound scanner.

FIG. 6 illustrates some embodiments of an example ultrasound system with a multi-functional ultrasound scanner.

FIG. 7 illustrates some embodiments of an example system with a multi-functional ultrasound scanner.

FIG. 8 illustrates some embodiments of an example system with a multi-functional ultrasound scanner.

FIG. 9 illustrates some embodiments of an example system with a multi-functional ultrasound scanner.

FIG. 10 illustrates some embodiments of an example system with a multi-functional ultrasound scanner.

FIG. 11 illustrates some embodiments of an example system for processing data from a multi-functional ultrasound scanner.

FIG. 12A illustrates some embodiments of example system components for use with a multi-functional ultrasound scanner.

FIG. 12B illustrates some embodiments of example system components for use with a multi-functional ultrasound scanner.

FIG. 13 illustrates some embodiments of an example system with a multi-functional ultrasound scanner.

FIG. 14 illustrates some embodiments of an example robotic system with a multi-functional ultrasound scanner.

FIG. 15 illustrates some embodiments of an example robotic system with a multi-functional ultrasound scanner.

FIG. 16 illustrates some embodiments of an example user interface of a system with a multi-functional ultrasound scanner.

FIG. 17 illustrates some embodiments of an example machine-learning architecture used to train a machine-learned model.

FIG. 18 illustrates some embodiments of an example machine-learned model using a convolutional neural network (CNN).

FIG. 19 illustrates some embodiments of an example device for use with a multi-functional ultrasound.

FIG. 20 illustrates some embodiments of a first example method.

FIG. 21 illustrates some embodiments of a second example method.

FIG. 22 illustrates some embodiments of a third example method.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Embodiments of the present invention include systems, devices, and methods for a multi-functional ultrasound scanner that includes an ultrasound array, at least one infrared sensor, and at least one microphone. In some embodiments, ultrasound data and infrared data are combined to form a composite image that depicts a needle during an ultrasound guided needle insertion procedure. The needle can be pre-heated or pre-cooled to emphasize its heat signature detectable via infrared radiation sensed by the infrared sensor. Additionally or alternatively, the ultrasound data can be combined with audio data sensed with a microphone on the multi-functional ultrasound scanner and directed to the patient anatomy imaged by the ultrasound array. In some embodiments, the system can uncover hidden correlations between the ultrasound data and the audio data and generate better inferences than conventional ultrasound systems. In some embodiments, the multi-functional ultrasound scanner included guidance systems to assist the operator in placing and orienting the scanner for imaging, and the multi-functional ultrasound scanner can communicate the guidance instructions to the user discreetly, so that they are not observable by other personnel in the environment, including the patient.

### Example Ultrasound Systems

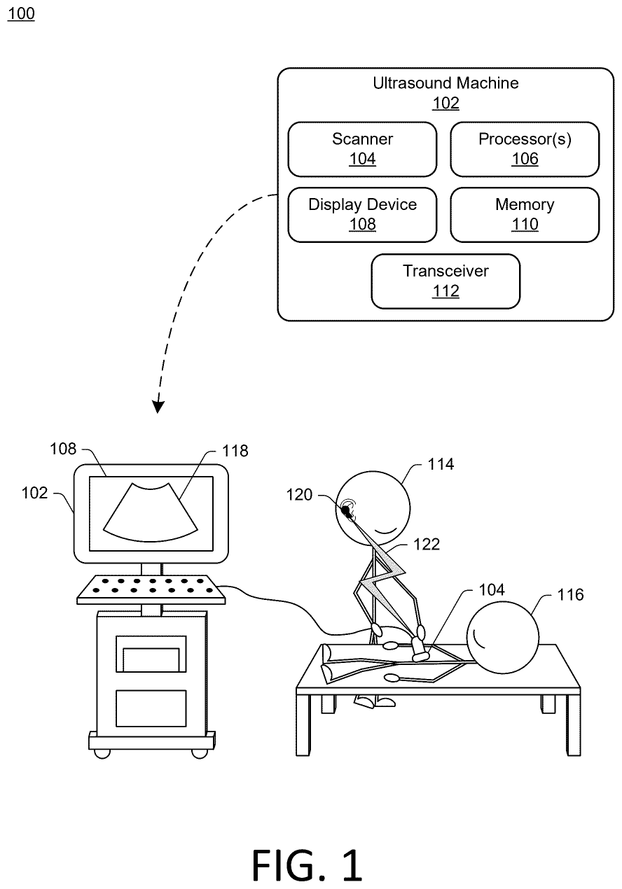

FIG. 1 illustrates some embodiments of an ultrasound system in an environment 100 with a multi-functional scanner during an ultrasound examination. The ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument (e.g., a needle). The ultrasound machine 102 includes various components, some of which include the scanner 104, one or more processors 106, a display device 108, a memory 110, and a transceiver 112.

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116, an interventional instrument, etc., for testing, diagnostic, therapeutic, or procedural reasons. In some embodiments, the scanner 104 includes an ultrasound transducer array and electronics communicatively coupled to the ultrasound transducer array to transmit ultrasound signals to the patient's anatomy and receive ultrasound signals reflected from the patient's anatomy. In some embodiments, the scanner 104 is an ultrasound scanner, which can also be referred to as an ultrasound probe or transducer. In some embodiments, the scanner 104 is a multi-array scanner. For instance, in some embodiments, a multi-array scanner includes one or more of the arrays described in U.S. patent application Ser. No. 18/613,694 filed on Mar. 22, 2024, entitled Multi-Dimensional and Multi-Frequency Ultrasound Transducers to Zhang et al., the disclosure of which is incorporated herein by reference in its entirety. In some embodiments, a multi-array scanner includes one or more of the arrays described in U.S. patent application Ser. No. 17/561,313 filed on Dec. 23, 2021, entitled Array Architecture and Interconnection for Transducers to Li et al., the disclosure of which is incorporated herein by reference in its entirety.

The display device 108 is coupled to the processor 106, which can include any suitable processor, number of processors, or processor system, such as one or more central processing units (CPUs), graphics processing units (GPUs), vector processors, reduced instruction set computing (RISC) processors, complex instruction set computing (CISC) processors, a very long instruction word (VLIW) processors, etc. The processor 106 can execute instructions stored on memory 110 to perform operations disclosed herein. For example, the processor 106 can process the reflected ultrasound signals to generate ultrasound data, including an ultrasound image. The display device 108 is configured to generate and display an ultrasound image (e.g., ultrasound image 118) of the anatomy and/or interventional instrument based on the ultrasound data generated by the processor 106 from the reflected ultrasound signals detected by the scanner 104. In some embodiments, the ultrasound data includes the ultrasound image 118 or data representing the ultrasound image 118. The transceiver 112 can be configured to transmit, e.g., over a network maintained by a care facility, the ultrasound data and/or any data related to the ultrasound examination, such as medical worksheet data, a health status report, etc., to a medical archiver (e.g., a vendor neutral archive (VNA)). In some embodiments, the transceiver 112 can receive data from the medical archiver, such as patient history data or previous examination data.

In some embodiments, the scanner 104 is a multi-functional ultrasound scanner that can include one or more sensors (that are different from the ultrasound transducer array of the scanner 104). For example, the scanner 104 can include a microphone (e.g., an audio sensor), infrared (IR) sensor to detect infrared radiation), a magnetic sensor, and the like. Sensor data captured by the sensor of the multi-functional ultrasound scanner can be communicated to the user 114 during the ultrasound examination. For example, the user 114 in FIG. 1 is wearing an earpiece 120 (e.g., a speaker placed into the user's 114 ear) and the sensor data can be communicated to the earpiece, in an audible fashion, over the communication link 122. The communication link 122 can implement a wired or wireless communication channel (e.g., Bluetooth™, Wi-Fi™, etc.) from the scanner 104 to the earpiece 120, and carry sensor data captured by a sensor of the scanner 104. For instance, the sensor of the scanner 104 can include a microphone and the sensor data can include audio data captured by the microphone. The microphone can be pointed (mechanically and/or electronically) at a patient anatomy, so that the user 114 can hear the audio of the patient anatomy (e.g., a heartbeat, breathing cycle, etc.) concurrently with the display of the ultrasound image 118 that visually depicts the patient anatomy. Accordingly, the multi-functional scanner 104 can implement a combined ultrasound scanner and stethoscope. In some embodiments, the ultrasound scanner 104 can communicate guidance instructions to the user 114 over the communication link 122 via the earpiece 120. The guidance instructions can discreetly communicate to the user 114 how to hold/orient the scanner 104, and the instructions can be imperceptible to the patient 116.

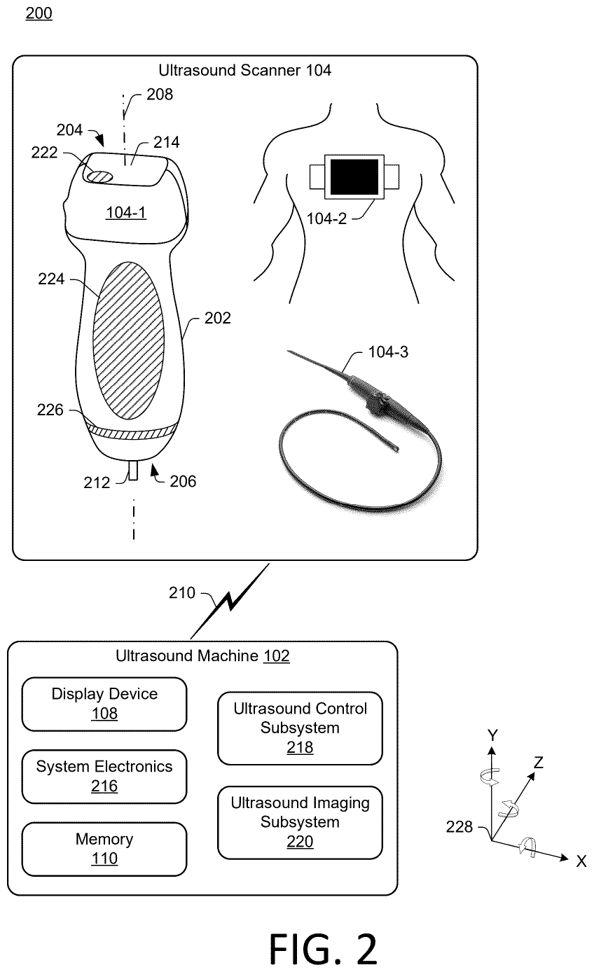

FIG. 2 illustrates an example implementation 200 of some embodiments of the ultrasound system illustrated in the environment 100 of FIG. 1. In the implementation 200, the scanner 104 (e.g., ultrasound scanner) can be any suitable type of ultrasound scanner. In some embodiments, the scanner 104 includes a scanner 104-1 configured for handheld operation, e.g., external to a patient's body. In some other embodiments, the scanner 104 includes scanner 104-2 which comprises a wearable ultrasound scanner (e.g., a patch-based ultrasound scanner) as described in U.S. patent application Ser. No. 18/957,403 filed on Nov. 22, 2024, entitled Determining Port Health with Ultrasound to Chamberlain et al., the disclosure of which is incorporated herein by reference in its entirety. For example, the scanner 104-2 can be worn by a patient during a procedure and/or for long term monitoring after the procedure. In still some other embodiments, the scanner 104 includes a scanner 104-3 configured for internal operation, e.g., for insertion into a patient, such as a transesophageal ultrasound scanner, as described in U.S. patent application Ser. No. 18/071,420 filed on Nov. 29, 2022, entitled A Transesophageal Ultrasound System to Nally et al., the disclosure of which is incorporated herein by reference in its entirety.

The scanner 104-1 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104-1. The scanner 104-1 is electrically coupled to an ultrasound imaging system (e.g., the ultrasound machine 102) via a coupling 210. In one example, the coupling 210 includes a cable that is attached to the proximal end portion 206 of the scanner 104-1 by a strain-relief element 212. In some embodiments, the coupling 210 includes a wireless coupling so that the scanner 104-1 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

The transducer assembly 214 can include multi-array transducer assemblies. For example, the transducer assembly 214 can include multi-array transducer assemblies that include any combination of piezoelectric micro-machined ultrasonic transducer (PMUT) array elements, lead zirconate titanate (PZT) array elements, and capacitive micro-machined ultrasonic transducer (CMUT) array elements. In some embodiments, the transducer assembly 214 includes at least one array with PZT array elements. Additionally or alternatively, the transducer assembly 214 includes a first array with array elements selected from the group consisting of PZT, PMUT, and CMUT array elements, and a second array with additional array elements selected from the group consisting of PZT, PMUT, and CMUT array elements. The elements of the first array can be of a different type than the elements of the second array (e.g., the first array can include PMUT elements and the second array can include CMUT elements). Alternatively, the elements of the first array can be of the same type as the elements of the second array (e.g., the first array and the second array can both include PMUT elements).

In some embodiments, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some embodiments, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receives electrical pulses from the scanner 104 that were created from the returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some embodiments, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In some embodiments, the disk storage device includes the memory 110, which is local to the ultrasound machine 102. Alternatively, the memory 110 used for storing the acquisition data can be remote, such as, for example, on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

In some embodiments, the ultrasound scanner 104 (e.g., ultrasound scanner 104-1) in the implementation 200 also includes one or more pressure sensors 222 on the lens of the scanner 104-1, and one or more pressure sensors 224 on the enclosure 202 of the scanner 104-1. The pressure sensors 222 and 224 can include in, on, or under a sensor region any suitable type of sensors for determining a pressure. In some embodiments, the pressure sensors 222 and 224 includes capacitive sensors that can measure a capacitance, or change in capacitance, caused by a user's touch or proximity of touch, as is common in touchscreen technologies. The pressure sensors 222 and 224 can generate pressure sensor data indicative of a touch or pressure. The pressure sensor data can include a binary indicator that indicates the presence and absence of a touch on the sensor. For instance, a “1” for pressure sensor data can indicate that a pressure is sensed at the pressure sensor, and a “0” for the pressure sensor data can indicate that a pressure is not sensed at the pressure sensor. Additionally or alternatively, the pressure sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity, and generally can be of any suitable shape and size, and generate pressure sensor data indicating pressure at any suitable number of points. For instance, in one example, the pressure sensors 222 cover an exterior surface of the lens of the scanner 104-1 and can be used to determine when the scanner is placed against a patient. Additionally or alternatively, the pressure sensors 224 can substantially cover the enclosure 202 of the scanner 104-1 and can be used to determine when a clinician grabs the scanner 104-1 for use in an ultrasound examination (e.g., the clinician has a suitable grip on the scanner 104-1 to perform the ultrasound examination). The ultrasound system can use the pressure sensor data from one or both of the pressure sensors 222 and 224 to generate a trigger signal that can be used by the ultrasound system. For instance, when the pressure sensor data from one or both of the pressure sensors 222 and 224 is above a threshold level, and/or the pressure sensor data from the pressure sensors 224 indicate a grip pattern indicative of a human operating the scanner, the system can generate a trigger signal. The trigger signal can be used to cause the ultrasound system to enable one or more machine-learned models to assist in a procedure using a multi-functional ultrasound scanner.

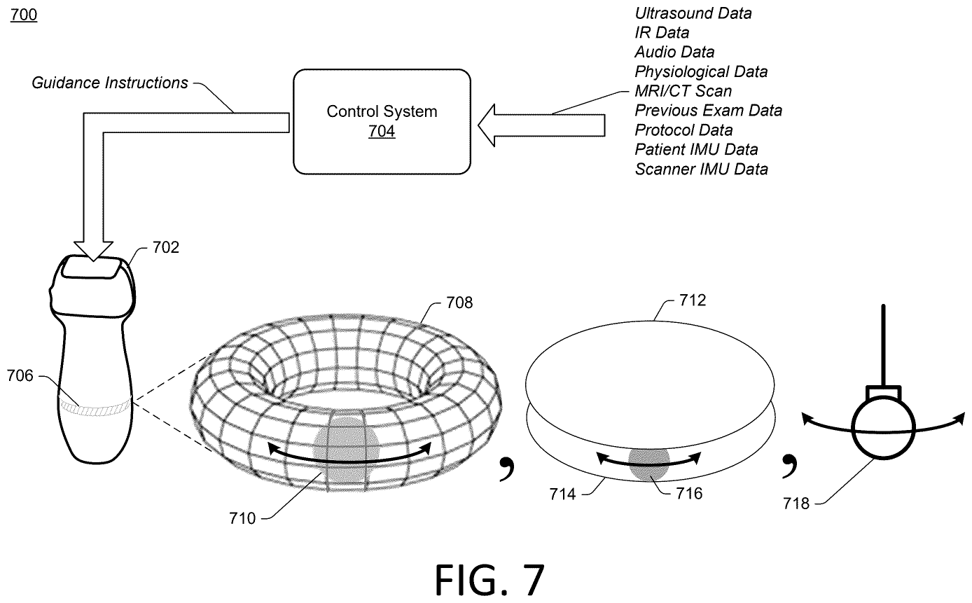

In some embodiments, pressure sensors 224 can also provide haptic feedback (e.g., vibrations, etc.) to a user (e.g., clinician). Such haptic feedback can be used to provide a clinician guidance and/or assistance, such as described in more described below in FIGS. 7-10 and its associated text.

In some embodiments, the ultrasound system uses the trigger signal to enable one or more light sources (e.g., microelectromechanical systems (MEMS) lasers) for needle insertion guidance. For instance, the light sources can indicate a current position of the needle tip, which array out of multiple arrays on the scanner the needle tip is positioned under, indicate the position of a blood vessel, indicate an insertion point for the needle on the patient's skin, etc. The light sources can project light onto the patient's skin.

In some embodiments, the scanner 104-1 includes an inertial measurement unit (IMU) 226 for generating positional data that determines a position and orientation of the scanner 104-1 in a coordinate system, e.g., the coordinate system 228 in FIG. 2. The IMU can include a combination of accelerometers, gyroscopes, and magnetometers, and generate positional data including data representing six degrees of freedom (6 DOF), such as yaw, pitch, and roll angles in the coordinate system. Typically, 6 DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera and fiducial markers on the scanner 104-1 (not shown in FIG. 2) to determine the positional data for the ultrasound scanner 104-1. In one example, the system generates, based on the positional data, a trigger signal as described above. For instance, the positional data can indicate that the scanner 104 is within a threshold distance of the patient, and the trigger signal can be used by the ultrasound system to enable one or more machine-learned models.

In some embodiments, the system includes an infrared sensor on an ultrasound machine, or elsewhere in the environment. The multi-functional ultrasound scanner can include a registration grid, such as a wrap, that is visible to the infrared sensor. During an ultrasound procedure, the infrared sensor can image the registration grid and infer the position and orientation of the scanner. Additionally or alternatively to using infrared, the system can use an optical camera and visible light as part of the registration.

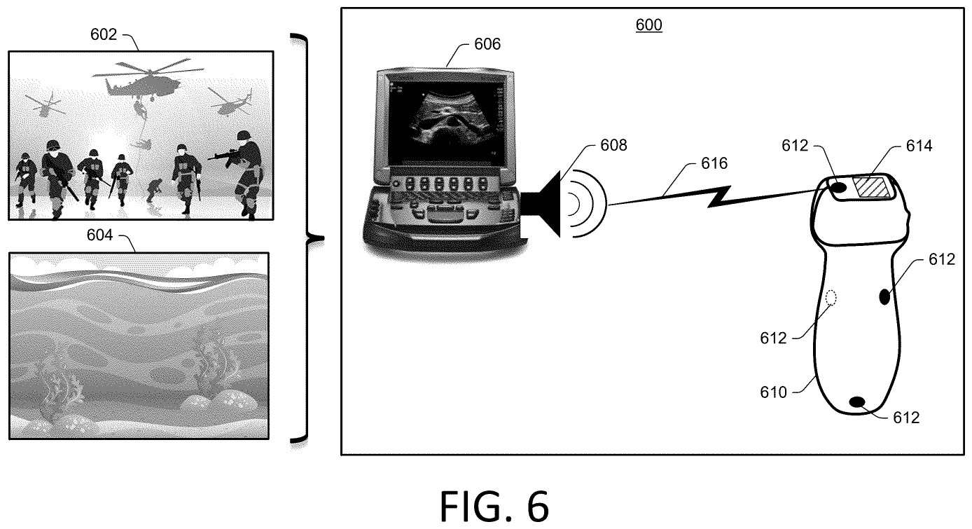

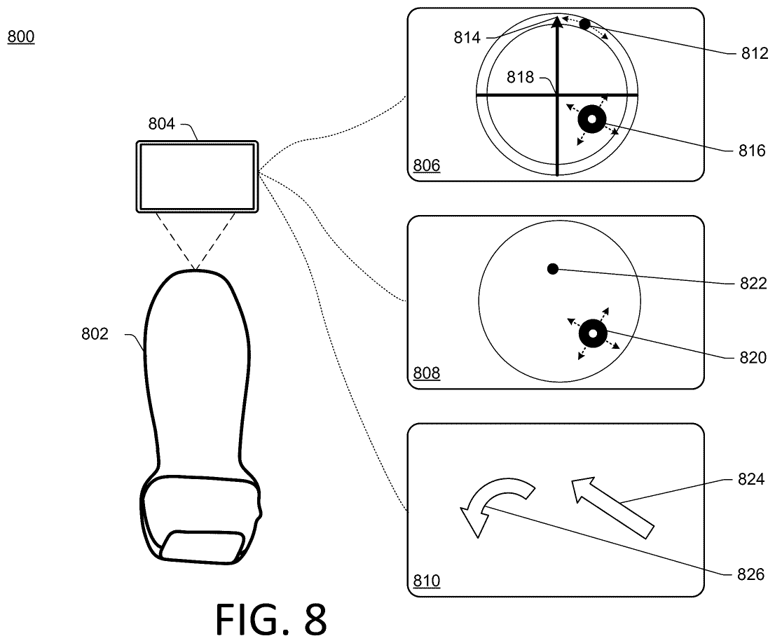

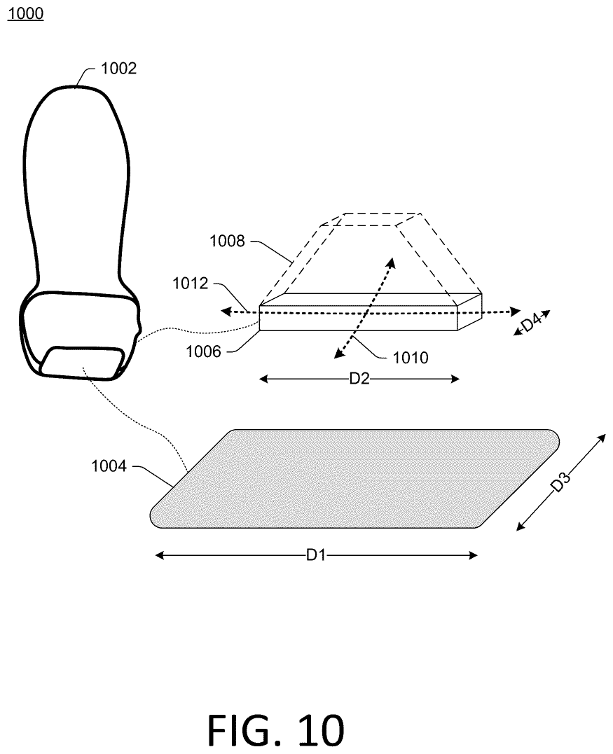

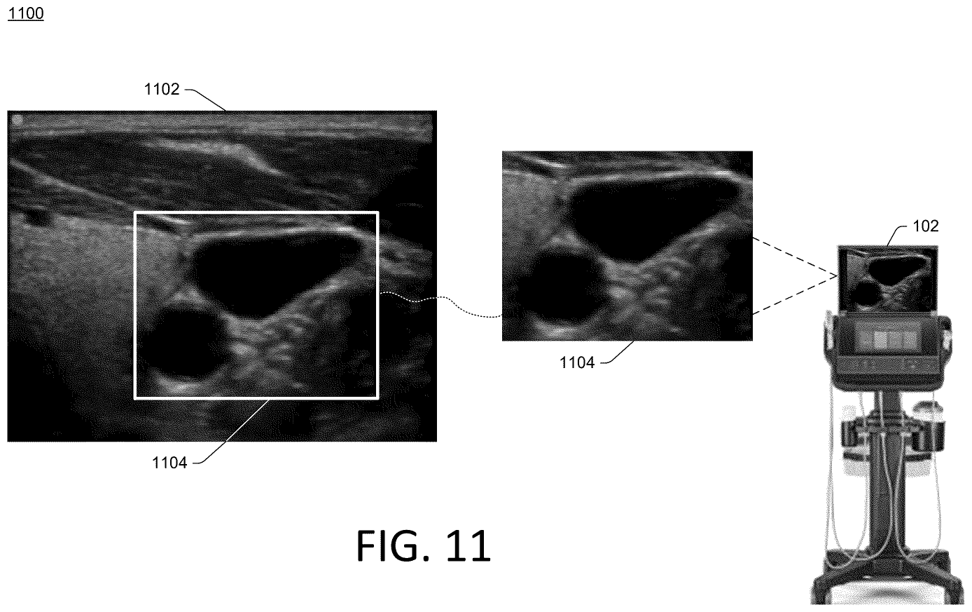

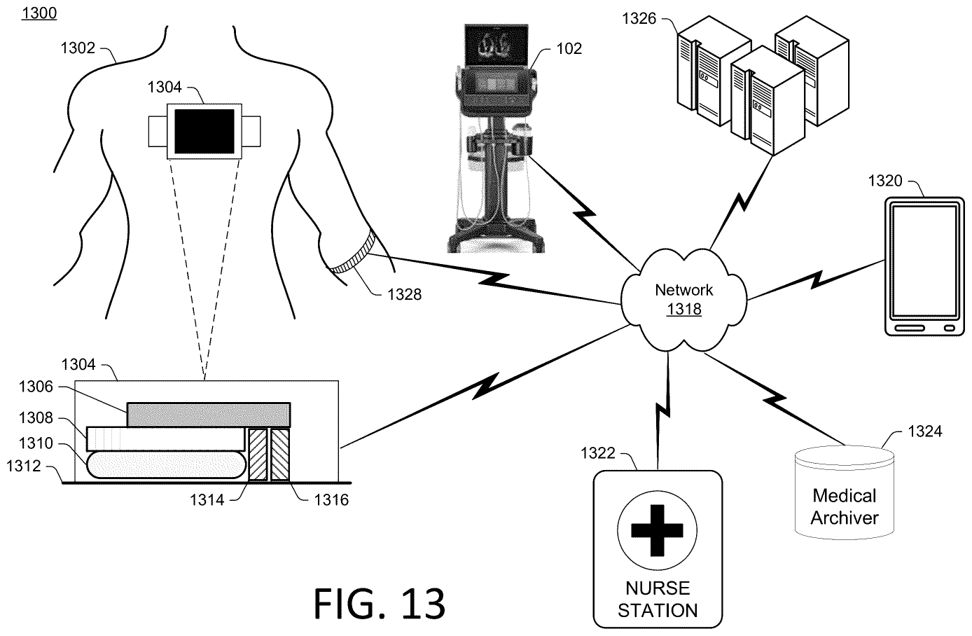

A trigger signal generated by the system, e.g., due to pressure data and/or positional data as described above, can be used to expedite the workflow of the system. In some embodiments, responsive to a trigger signal, the system automatically requests a patient history, such as from a medical archiver (e.g., a VNA). The patient history can include previously-generated health status reports for the patient, which can be displayed by the system for comparison against a current health status report. Further, a trigger signal generated by the system, e.g., due to pressure data and/or positional data as described above, can be used to determine an insertion location on the patient (e.g., a needle insertion location), and this location can be identified by projecting a light from the ultrasound scanner onto the patient. Other example uses of the trigger signal include, but are not limited to:
1) Run a calibration routine on the multi-functional ultrasound scanner to check for faults (e.g., array element failures);
2) Enable a gel wiper on the multi-functional ultrasound scanner;
3) Configure and/or enable one or more ultrasound arrays of the multi-functional scanner ultrasound scanner;
4) Configure and/or enable one or more sensors (e.g., infrared sensors, microphones, magnetic sensors, cameras, etc.) of the multi-functional ultrasound scanner;
5) Generate an image quality score (e.g., with a machine-learned model) for an image generated by the system;
6) Replace the ultrasound array with a different ultrasound array having another pitch (can be based on image quality score);
7) Enable data capture of physiological patient parameters, which can be used to gate/synchronize the ultrasound data;
8) Enable access to a quantum computer to request a data format, as described in U.S. patent application Ser. No. 19/007,231 filed on Dec. 31, 2024 entitled Quantum-Processed Ultrasound to Endres et al., the disclosure of which is incorporated herein by reference in its entirety;
9) Change the ultrasound scanner to a different ultrasound scanner, e.g., in a robotic system that includes multiple robotic controllers with multiple ultrasound scanners;
10) Replace a replaceable transducer assembly of an ultrasound scanner with another replaceable transducer assembly;
11) Enable a needle visualization algorithm, e.g., a machine-learned model;
12) Enable a processor system to generate a composite image from ultrasound data and other sensor data, e.g., infrared data, audio data, and the like;
13) Enable the temperature adjustment of a needle (e.g., as described in more detail with respect to FIG. 13). For example, the system can activate one of the packages 1210, 1212;
14) Enable a projector on the multi-functional ultrasound scanner (e.g., a MEMS projector) to project an image (e.g., an infrared image) onto the patient;
15) Enable the system to generate a visual representation of an audio signal, such as a spectrogram, a time-frequency distribution, a Wigner distribution, and the like;
16) Enable a machine-learned model to generate an inference using an ultrasound image as input at a top layer of the machine-learned model and at least one of infrared data and audio data as conditional input at another layer of the machine-learned model;
17) Enable the multi-functional ultrasound scanner to receive commands from an ultrasound machine via an audio communication link that communicates audio from a speaker of the ultrasound machine to a microphone of the multi-functional ultrasound scanner (as described in more detail with respect to FIG. 6);
18) Enable a guidance mechanism of the multi-functional ultrasound scanner, e.g., to directionally modulate a weight of the multi-functional ultrasound scanner (as described in more detail with respect to FIG. 7) and/or enable a display device of the multi-functional ultrasound scanner (e.g., as described in more detail with respect to FIG. 8);
19) Enable an adaptive mount of the multi-functional ultrasound scanner to adjust its position to adjust the location of at least one of an ultrasound transducer array, an infrared sensor, and a microphone relative to a lens of the multi-functional ultrasound scanner (e.g., as described in more detail with respect to FIG. 10); and
20) Enable a region of the ultrasound data to be displayed on an ultrasound machine, and to adjust the region over time (e.g., as described in more detail with respect to FIG. 11).

The scanner 104 also includes the example scanner 104-2 that is coupled to the ultrasound machine via the coupling 210, e.g., via a wireless connection. The scanner 104-2 is an example of a wearable ultrasound scanner that can be patient worn, such as, for example, a patch that can be placed over a patient's anatomy during a procedure or for monitoring after the procedure. For instance, the scanner 104-2 can be placed on a patient for longer term monitoring, e.g., days, weeks, or months, to continuously (e.g., periodically) monitor the effects of a procedure.

In some embodiments, the scanner 104 also includes the example scanner 104-3 that is coupled to the ultrasound machine via the coupling 210, e.g., via a wired/cabled connection. The scanner 104-3 is an example of an ultrasound scanner configured for internal insertion into a patient, to internally scan the patient. For instance, the scanner 104-3 can include a transesophageal ultrasound scanner inserted orally into a patient and through their esophagus to scan anatomy from an internal viewpoint, including cardiac or respiratory anatomy. The scanner 104-3 can include multiple sensors, e.g., microphones, infrared sensors, and the like, in addition to one or more ultrasound transducer arrays. The sensor data from the sensors, e.g., infrared data from an infrared sensor of the scanner 104-3, can be used to provide additional imaging (e.g., other than ultrasound image data) internal to a patient during a procedure. This additional imaging can increase the accurate placement of an interventional instrument during the procedure, such as a cutter, cauterizer, robotic arm for moving organs, etc., thereby reducing the damage done to the patient during the procedure compared to conventional systems, such as systems that provide little imaging, imaging from a limited viewpoint, or systems that provide no imaging (e.g., a blind procedure). In some embodiments, the sensor data (e.g., infrared data) can be focused in a direction that is different from a direction the ultrasound is focused. Additionally or alternatively, the sensor data (e.g., infrared data) can be focused in a similar direction as the ultrasound is focused, so that the infrared data and ultrasound data can be combined together, e.g., to form a three-dimensional (3D) composite image of the patient anatomy. In some embodiments, the system generates the 3D image in real time, e.g., without perceptible delay to the user.

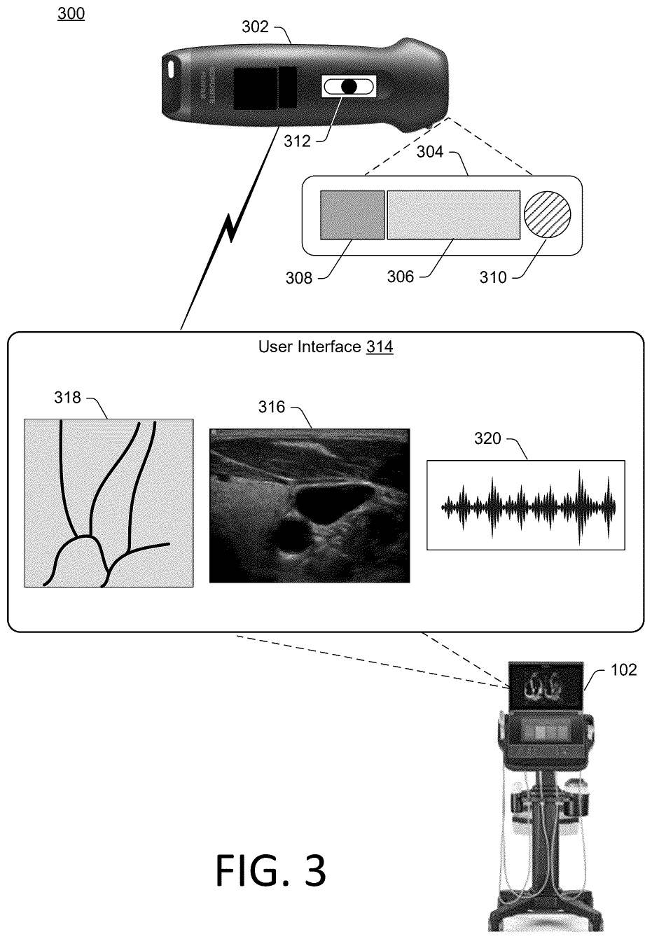

As described above, the scanner 104, e.g., the scanner 104-1, the scanner 104-2, and/or the scanner 104-3 can be a multi-functional ultrasound scanner that generates ultrasound imaging data with an ultrasound transducer array and at least one other type of sensor data from a sensor of the scanner 104. In some embodiments, the sensor data (e.g., infrared data, audio data, magnetic data, etc.) is captured by the scanner 104 concurrently with the ultrasound data. Hence, the sensor data and the ultrasound data can be correlated. As an example of a multi-functional scanner, FIG. 3 illustrates an example ultrasound system 300 with a multi-functional ultrasound scanner 302. The multi-functional ultrasound scanner 302 is an example of the scanner 104, e.g., the scanner 104-1, the scanner 104-2, and/or the scanner 104-3.

In some embodiments, the multi-functional ultrasound scanner 302 includes a lens 304 at its distal end portion (204 in FIG. 2), and the lens 304 covers an ultrasound transducer array 306. The lens 304 also covers multiple sensors of the multi-functional ultrasound scanner 302. In some embodiments, the sensors of the multi-functional ultrasound scanner 302 include an infrared sensor 308 and a microphone 310. Other examples of sensors that can be included in the multi-functional ultrasound scanner 302 include a magnetic sensor (e.g., implemented to detect and measure a magnetic field generated by a patient anatomy), a thermometer implemented to measure temperatures of patient anatomies, a camera implemented to capture optical light, etc. The infrared sensor 308 and the microphone 310 are meant to be exemplary and non-limiting examples of sensors that can be included in a multi-functional ultrasound scanner for improved imaging compared to conventional ultrasound systems that do not include a multi-functional ultrasound scanner 302.

The infrared sensor 308 can be implemented to receive infrared radiation and generate infrared data (e.g., heat data) based on the infrared radiation. In some embodiments, the infrared sensor 308 receives infrared radiation generated by a patient anatomy. For instance, different organs can emit different frequencies and profiles of infrared radiation, which can be received by the infrared sensor 308 and used by the system to generate an infrared image that depicts the different organs (e.g., patient anatomies). Additionally or alternatively, the infrared sensor 308 can include an infrared transmitter than transmits infrared radiation into a patient. The infrared sensor 308 can include an infrared receiver to receive reflections of the infrared radiation from a patient anatomy, and the system can determine infrared imaging data based on the received infrared radiation.

The microphone 310 can be implemented to receive audio emitted by the patient anatomy. For example, different organs can emit different sounds (e.g., frequencies and audio profiles). The system can use the audio data received by the microphone 310 to infer data about the organs. For instance, the system can implement a machine-learned model to process the audio data and generate a score for the patient anatomy. A low score (e.g., two out of ten) can indicate an unhealthy patient anatomy, and a high score (e.g., nine out of ten) can indicate a healthy patient anatomy.

In some embodiments, the multi-functional ultrasound scanner 302 includes a control 312 (indicated in FIG. 3 as a multi-position switch) that can be configured to enable any suitable combination of the ultrasound transducer array 306, the infrared sensor 308, and the microphone 310. In some embodiments, the control 312 includes a setting to enable simultaneous operation of the ultrasound transducer array 306, the infrared sensor 308, and the microphone 310. In some other embodiments, the control 312 includes a setting to enable simultaneous operation of the ultrasound transducer array 306 and the infrared sensor 308. In yet some other embodiments, the control 312 can include a setting to enable simultaneous operation of the ultrasound transducer array 306 and the microphone 310.

Additionally or alternatively, in some embodiments, the ultrasound machine 102 can include a user interface 314 that can include one or more control options (not shown for clarity) to enable and/or disable one or more of the sensors of the multi-functional ultrasound scanner 302, including the ultrasound transducer array 306, the infrared sensor 308, and the microphone 310. In some embodiments, the user interface 314 can also display images generated by the system based on data gathered by the ultrasound transducer array 306, the infrared sensor 308, and/or the microphone 310. For example, the user interface 314 includes an ultrasound image 316 generated from ultrasound data received by the ultrasound transducer array 306. The user interface 314 also includes an infrared image 318 generated from infrared radiation received by the infrared sensor 308. In some embodiments, the user interface 314 also includes a visual representation 320 of an audio signal captured received by the microphone 310. The visual representation 320 can include any suitable representation of an audio signal, such as a plot of the amplitude of the audio signal versus time, a spectrogram, a time-frequency distribution, a Wigner distribution, and the like.

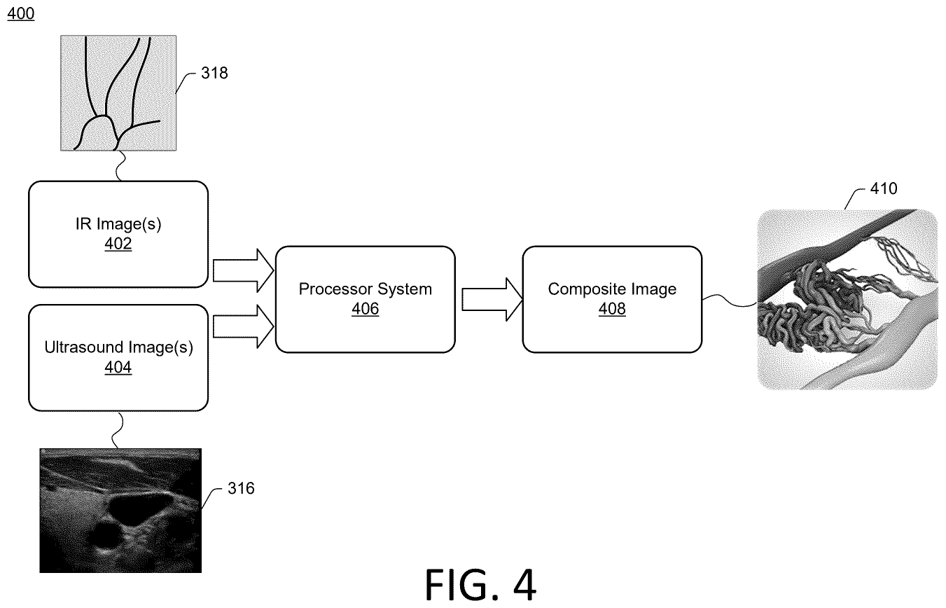

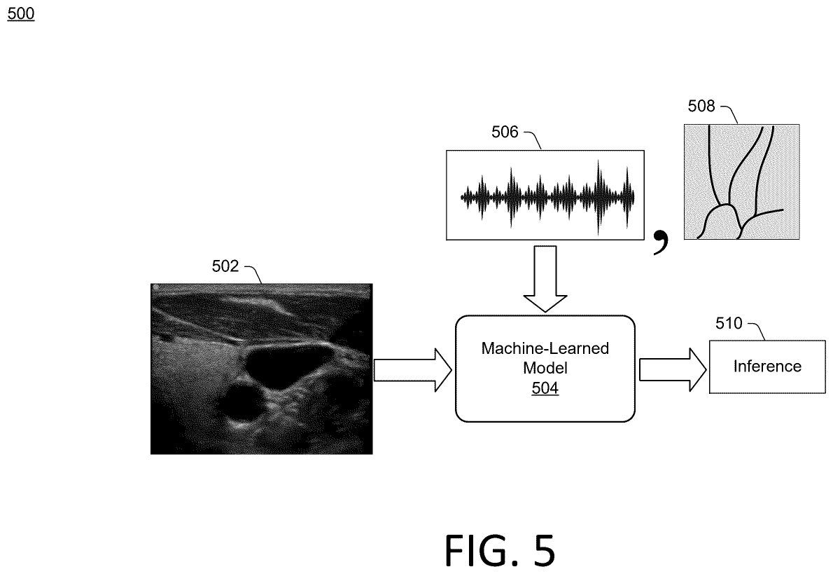

In some embodiments, the ultrasound transducer array 306, the infrared sensor 308, and the microphone 310 can be mechanically and/or electronically steered or otherwise positioned within the multi-functional ultrasound scanner 302 so that they are focused on a same patient anatomy. In this case, the data from the different sensors of the multi-functional ultrasound scanner 302 is correlated, and can be concurrently processed (e.g., with a machine-learned model) to uncover the correlations and/or generate composite data (described below in more detail with respect to FIGS. 4 and 5).

In some embodiments, the user interface 314 displays two or more of the ultrasound image 316, the infrared image 318, and the visual representation 320 simultaneously. Additionally or alternatively, the user interface 314 can display two or more of the ultrasound image 316, the infrared image 318, and the visual representation 320 sequentially. For example, in some embodiments, the infrared image 318 depicts a vascular architecture in a C-plane, and the user interface 314 can display the infrared image 318 before display of the ultrasound image 316. For instance, during an ultrasound-guided needle insertion procedure into the vascular architecture, the user interface 314 can display the infrared image 318 while the needle is outside a threshold distance of a vein or artery. When the system detects that the needle is within the threshold distance of the vein or artery, the system can then display the ultrasound image 316 (e.g., a B-mode image). When the ultrasound image 316 is displayed, the system can cease display of the infrared image 318. Alternatively, the system can maintain display of the infrared image 318 while the ultrasound image 316 is displayed. Hence, the system can provide the user a view of the needle throughout the needle insertion procedure. In one example, when the system detects that the needle is within the threshold distance of the vein or artery, the system then displays a composite image generated by the system based on the ultrasound image 316 and the infrared image 318. The composite image can include a 3D image.

In some embodiments, the system projects one or more of the ultrasound image 316, the infrared image 318, and the visual representation 320 onto the patient (e.g., with a MEMS projector of the multi-functional ultrasound scanner 302, not shown for clarity), and displays another of the ultrasound image 316, the infrared image 318, and the visual representation 320 on the user interface 314. For instance, the multi-functional ultrasound scanner 302 can project the infrared image 318 onto a patient's arm, and the user interface 314 can simultaneously display the ultrasound image 316.

In some embodiments, the system determines an inference of the patient anatomy from one of the ultrasound image 316, the infrared image 318, and the visual representation 320. The inference can include a label, classification, segmentation, etc. The system can then augment another of the ultrasound image 316, the infrared image 318, and the visual representation 320 to indicate the inference. For example, the system can process the ultrasound image 316 with a machine-learned model to classify a blood vessel a vein or artery, and then label the blood vessel with a “V” for vein or “A” for artery in the infrared image 318, or color-code the blood vessels in the infrared image 318 to denote their classifications.

In some embodiments, the system can process one or both of the infrared image 318 and the visual representation 320 to determine an imaging parameter, and configure the ultrasound system 102 so that the ultrasound image 316 is generated according to the imaging parameter. For example, in some embodiments, the system can determine a depth of a blood vessel in the infrared image 318, set an imaging depth and/or gain of the ultrasound machine 102 to image the depth of the blood vessel, and then capture the ultrasound image 316 with the ultrasound machine configured with the imaging depth and/or gain.

In some embodiments, the multi-functional ultrasound scanner 302 includes a wiper (not shown in FIG. 3 for clarity), e.g., a mechanical blade configured to move ultrasound gel (e.g., acoustic coupling agent) away from one of the sensors, such as away from the infrared sensor 308 and/or the microphone 310, and towards the ultrasound transducer array 306. The wiper can be activated/enabled based on movement of the multi-functional ultrasound scanner 302 relative to the patient, and triggered by IMU data of the multi-functional ultrasound scanner 302. In some embodiments, the wiper is activated/enabled responsive to a trigger signal generated based on pressure sensor data, as previously described. Additionally or alternatively, in some embodiments, the infrared sensor 308 is physically separated from the ultrasound transducer array 306 (e.g., there can be a space, or gap, between the infrared sensor 308 and the ultrasound transducer array 306 as they are positioned on the multi-functional scanner 302). Hence, the gel can be placed under the ultrasound transducer array 306, and not under the infrared sensor 308. In some embodiments, the system includes a compensation system to adjust the infrared data based on it being affected by the ultrasound gel. For instance, the compensation system can adjust a gain of the infrared data based on an attenuation caused by the ultrasound gel. Different gel types can have different attenuations, so the compensation system can adjust the gain of the infrared data based on the type of ultrasound gel used.

In some embodiments, the system can adjust the audio signal captured by the microphone 310 based on ultrasound captured by the ultrasound transducer array 306. Adjusting the audio signal can include modulating the playback speed of the audio signal, e.g., to match a frame rate of the ultrasound so that the ultrasound images are displayed at a same rate as the audio is played for the user. In some embodiments, adjusting the audio signal can include adding information to the audio signal, including additional audio data. For instance, if the audio signal includes data from one chamber of a heart, the system can generate additional audio for another chamber of the heart and combine the additional audio with the audio signal. Combining here can include appending the additional audio so that it plays at a different time, e.g., sequentially to the audio signal. Additionally or alternatively, combining can include adding the additional audio to the audio signal so that they both are played back together, at the same time. In some other embodiments, the system adds systole audio data to a diastole audio signal captured by the microphone 310. The audio added to the audio signal captured by the microphone 310 can help the user direct the scanner 302 to a patient anatomy. For instance, the added audio can represent the desired patient anatomy, and help the user target this patient anatomy. In some embodiments, the added audio acts as a target to the user. For instance, the user can adjust the scanner until the audio signal generated by the microphone 310 matches the added audio. In this way, the added audio acts as a guide to direct the user to scan the desired patient anatomy.

In some embodiments, the system implements a noise cancellation system to remove noise on the audio signal captured by the microphone 310 caused by vibrating elements (e.g., PZT) of the ultrasound transducer array 306. For example, PZT elements can emit sound in the audible spectrum that can corrupt the audio signal captured by the microphone 310. The noise cancellation system can be adaptive. For instance, the noise cancellation system can capture sound emitted by the PZT elements (e.g., with an additional microphone) and use this captured sound as a reference signal to the noise cancellation system. The noise cancellation system can use this reference signal to adaptively, e.g., iteratively, to remove the noise from the PZT elements that corrupt the audio signal captured by the microphone 310.

FIG. 4 illustrates some embodiments of an example ultrasound system 400 for processing data from a multi-functional ultrasound scanner. Referring to FIG. 4, the system 400 includes infrared images 402. The infrared image 318 is an example of the infrared images 402. The system 400 also includes ultrasound images 404. The ultrasound image 316 is an example of the ultrasound images 404. The system 400 also includes a processor system 406 configured to generate a composite image 408. The processor(s) 106 in FIG. 1 is an example of the processor system 406. The composite image 410 is an example of the composite image 408 corresponding to the infrared image 318 and the ultrasound image 316.

The processor system 406 can implement any suitable processing algorithm to generate the composite image 408. In some embodiments, the processor system 406 implements an edge detection algorithm to generate the composite image 408. Additionally or alternatively, the processor system 406 can implement one or more machine-learned models to generate the composite image 408. Additionally or alternatively, the processor system 406 can implement one or more multi-planar 3D image reconstruction algorithms to generate the composite image 408.

Further, the composite image 408 can be generated by combining any suitable data in, or generated from, the infrared images 402 and the ultrasound images 404. In some embodiments, the composite image 408 includes a 3D image. For instance, the infrared images 402 and the ultrasound images 404 can include different imaging planes that can be combined to form a 3D image. In some embodiments, the imaging plane of the infrared images 402 is orthogonal to the imaging plane of the ultrasound images 404. Since the tissue characteristics of different organs can vary (e.g., different tissues have a different wavelength responses), combining the infrared images 402 and the ultrasound images 404 can allow for more distinct organ rendering compared to infrared imaging or ultrasound imaging alone.

In some embodiments, the system can generate one or more composite images 408 in real time (e.g., without perceptible delay to a user). Hence, for an ultrasound-guided needle insertion procedure, the system can achieve real-time, 3D rendering that illustrates the needle movement as the clinician inserts the needle, and tracks the needle with the 3D rendered view via the composite images 408. In some embodiments, the system has access to a quantum computer to request a data format, as described in U.S. patent application Ser. No. 19/007,231 filed on Dec. 31, 2024 entitled Quantum-Processed Ultrasound to Endres et al., the disclosure of which is incorporated herein by reference in its entirety. The system can then format the infrared images 402 and the ultrasound images 404 according to the data format, and send the formatted images to the quantum computer. The quantum computer can implement a quantum machine-learned model to evolve a quantum state based on the formatted images and generate a quantum composite image. The processor system 406 can receive from the quantum computer the quantum composite image and format it into a format viewable by the ultrasound machine 102 to form the composite image 408.

FIG. 5 illustrates some embodiments of an example ultrasound system 500 for processing data from a multi-functional ultrasound scanner. Referring to FIG. 5, the system 500 receives an ultrasound image 502 as a primary input to a machine-learned model 504 implemented to generate an inference 510 for the ultrasound image 502. Non-limiting examples of the inference 510 include a label, classification, segmentation, region of interest, and the like. The ultrasound image 502 is an example of an ultrasound image that can be generated based on ultrasound received by the ultrasound transducer array 306 of a multi-functional ultrasound scanner.

The system 500 also receives audio data 506 and an infrared image 508 as secondary, or conditional inputs, to the machine-learned model 504. The audio data 506 is an example of data that can be generated based on audio received by the microphone 310 of a multi-functional ultrasound scanner. The audio data 506 can be of any suitable data format, including one dimensional data, such as an audio signal captured over a time interval, and/or two dimensional image data, such as a spectrogram, a time-frequency distribution, a Wigner distribution, the visual representation 320, and the like.

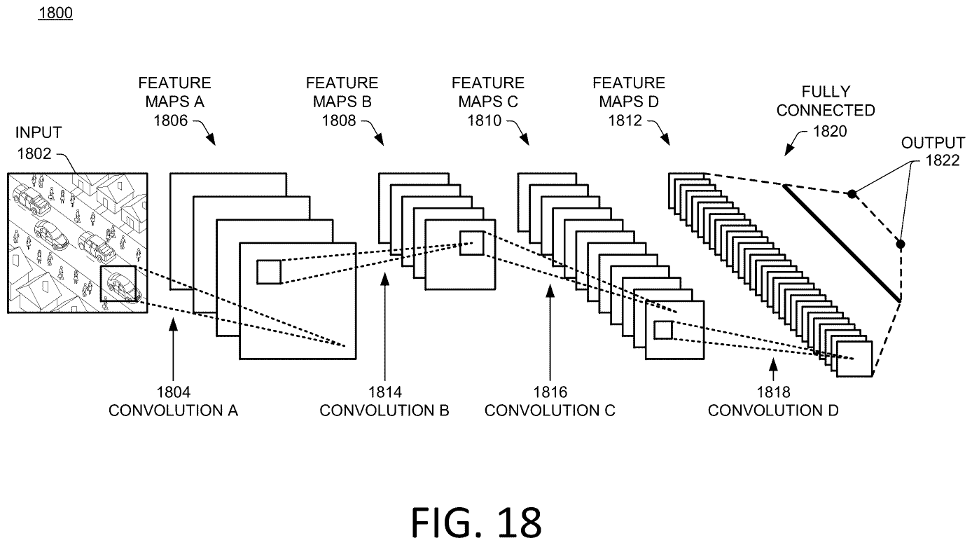

In some embodiments, the machine-learned model 504 processes the ultrasound image 502 as a primary input at its top level. For instance, referring to FIG. 18, the machine-learned model 504 processes the ultrasound image with convolution A 1804, convolution B 1814, and convolution C 1816 of the convolutional neural network depicted in FIG. 18 to generate a feature map C 1810 for the ultrasound image 502. However, since the audio data 506 and an infrared image 508 can be processed as secondary, or conditional inputs to the machine-learned model 504, in some embodiments the machine-learned model 504 does not process the audio data 506 and/or the infrared image 508 at its top levels, but instead can append the secondary data to an internal feature map it generated for the ultrasound image 502. For instance, the machine-learned model 504 can append the audio data 506 and/or the infrared image 508 to the feature map C 1810 generated for the ultrasound image 502. The machine-learned model 504 can then process the feature map C 1810 together with the secondary/conditional data appended to it by other layers of the convolutional neural network, such as with the convolution D 1818, to generate the output 1822. Hence, the secondary/conditional input data, e.g., the audio data 506 and an infrared image 508, can be processed by fewer layers of the machine-learned model 504 than the primary input, e.g., the ultrasound image 502.

Returning to FIG. 5, in some embodiments, the audio data 506 and/or the infrared image 508 can be acquired simultaneously with the ultrasound data represented in the ultrasound image 502. Hence, the audio data 506 and/or the infrared image 508 can be correlated with the ultrasound image 502. For instance, the audio data 506 of a patient anatomy can contain information that is not exploited when only using ultrasound image data to generate inferences, such as respiratory, cardiac, organ, and vascular audio profiles. Using the audio signal 506 and/or the infrared image 508 as a secondary/conditional input enables the machine-learned model 504 to determine the hidden correlations between the audio and/or IR data and the ultrasound image data, providing better inference generation compared to systems that solely process ultrasound image data. For instance, an unhealthy organ may have a distinct audio signature, such as a gurgle in respiratory audio data. In another example, an unhealthy organ may have a distinct IR signature, such as hot spot in a heat map (e.g., infrared image 508) of the organ generated based on the infrared data.

FIG. 6 illustrates some embodiments of an example ultrasound system 600 with a multi-functional ultrasound scanner. Referring to FIG. 6, the system 600 can be used in a clinical environment, such as the environment 100 depicted in FIG. 1. Further, the system 600 can be used in the environments 602 and 604. The environment 602 depicts an environment in which electromagnetic radiation is undesirable, such as a role-1 military environment. For instance, electromagnetic radiation can be detected by an enemy's drones, or otherwise attract unwanted attention in a military environment. The environment 604 depicts an underwater environment, e.g., for marine life veterinary care. In this underwater environment, electromagnetic (EM) radiation propagates poorly, which can prevent the use of conventional wireless ultrasound scanners and instead force the use of wired/cabled ultrasound scanners, which can be prone to leakage due to cable seals, and encumbrances due to the dynamic underwater environment.

Accordingly, in some embodiments, the system 600 includes an ultrasound machine 606 (e.g., a portable, point-of-care ultrasound machine) that includes an audio speaker 608. The system 600 also includes an ultrasound scanner 610, e.g., a multi-functional ultrasound scanner. As such, the scanner 610 includes at least one microphone 612 and an ultrasound array 614. The scanner 610 and the ultrasound machine 606 are coupled via an audio communication link 616, which conveys audio from the speaker 608 to at least one microphone 612. The audio on the audio communication link 616 can be encoded with command and control data to control the scanner 610, such as to enable arrays of a multi-array transducer, set imaging mode configuration, set a transmit and/or receive frequency, etc. In some embodiments, the audio communication link 616 operates outside the human-perceptible frequency range, e.g., 28-32 KHz. Because the audio communication link 616 emits no EM radiation, it is suitable to the environment 602. For instance, it can be more difficult to detect than an RF signal. Further, an audio signal propagates well under water, unlike radio frequencies or EM radiation, so that the audio communication link 616 is suitable to the environment 604.

In some embodiments, the audio communication link 616 is one directional, e.g., to communicate data from the ultrasound machine 606 to the scanner 610. In some other embodiments, the audio communication link 616 is two directional. In this case, the scanner 610 can include a speaker (not shown in FIG. 6) that can communicate data over the audio communication link 616 to a microphone of the ultrasound machine 606 (not shown in FIG. 6). In some embodiments, the data over the audio communication link 616 is encoded to target the scanner 610, to avoid the data being received and encoded by another device in the environment, e.g., another multi-functional ultrasound scanner.

In some embodiments, the microphone(s) 612 of the scanner 610 includes multiple microphones. In some embodiments, the microphone(s) 612 of the scanner 610 is implemented to detect audio caused by tactile gestures on the ultrasound scanner 610, such as user taps on the enclosure of the scanner 610. The gestures (e.g., taps) can be performed by a user of the scanner 610 to assist in workflow and/or reporting. In some embodiments, based on a tactile gesture (e.g., tap, swipe, etc.) detected by the microphone(s) 612, the system 600 configures the ultrasound machine 606 to start or end recording, adjust a gain, adjust a depth, etc. In an example, the operator can tap or swipe on the right side of the scanner 610 to adjust gain (e.g., tap means adjust higher, swipe means adjust lower), and tap/swipe on the left side of the scanner 610 to adjust a depth, etc.

In some embodiments, the system 600 receives a voice command from a user, e.g., via the microphone(s) 612, and the user can confirm the voice command by performing a tactile gesture (e.g., tap/swipe) that is sensed via the microphone(s) 612. In some embodiments, the system 600 implements a compensation system (e.g., an adaptive noise canceller) to cancel interference from the tactile gestures that can leak onto and affect the ultrasound signal received by the transducer array 614. In some embodiments, the system 600 is implemented to edit a medical worksheet based on the audio caused by the tactile gestures. Additionally or alternatively, the system 600 can configure, based on the audio caused by the tactile gestures, the transducer array 614 to transmit ultrasound towards a patient anatomy and receive reflections of the ultrasound from the patient anatomy.

In some embodiments, audio detected by microphone 612 next to transducer array 614 can be filtered to remove noise associated with movement of the ultrasound scanner 610 against a patient. Such filtering can be done using a noise profile associated with noise associated with movement of the ultrasound scanner 610 against a patient.

In some embodiments, microphone(s) 612 can be turned on and/or off based on grip data associated with the ultrasound scanner 610. For example, pressure sensors (e.g., pressure sensors 224 of FIG. 2) can be used to capture pressure placed by the user on the ultrasound scanner 610 to turn on and off one or more of microphone(s) 612.

### Example Guidance/Assistance

Many ultrasound systems include guidance mechanisms to assist a clinician, like a graphic displayed on an ultrasound machine that indicates a probe placement to achieve an image view, or a quality indicator showing feedback on how good the currently obtained image is. These mechanisms, when enabled, can usually be viewed by other personnel in the room. Many clinicians, however, do not enable the guidance mechanisms out of ego, fear etc., which is exacerbated when their use can be witnessed by the other personnel. Hence, clinicians may not utilize the ultrasound system to its fullest capabilities and may capture sub-optimal ultrasound images. Accordingly, in some embodiments, a multi-functional scanner includes and/or supports a guidance system to discreetly convey instructions to a user. For example, as described previously with respect to FIG. 1, the ultrasound scanner 104 can communicate guidance instructions to the user 114 over the communication link 122 via the earpiece 120 so that the patient 116 does not perceive the instructions.

In another example, FIG. 7 illustrates some embodiments of an example system 700 with a multi-functional ultrasound scanner in accordance with the present invention. The system 700 can be used to discreetly provide guidance to a user, and the guidance can be withheld from others in the environment, such as the patient. The system 700 includes an ultrasound scanner 702, e.g., a multi-functional ultrasound scanner as described herein. The scanners 104, 302, 610 are examples of the ultrasound scanner 702. The system 700 also includes a control system 704, which can be implemented to generate guidance instructions for the ultrasound scanner 702 based on any suitable input. In some embodiments, the control system 704 receives data from the ultrasound scanner 702 to generate the guidance instructions. For example, the control system 704 can include ultrasound data generated from one or more ultrasound transducer arrays (e.g., the array 306), infrared data (e.g., from the infrared sensor 308), and/or audio data (e.g., from the microphone 310). In some embodiments, the control system 704 implements one or more machine-learned models to process the input signals and generate the guidance instructions. For instance, the machine-learned model can identify a patient anatomy from the input data, and generate guidance instructions that indicate how to hold/orient the scanner 702 to image the patient anatomy with a desired quality, e.g., to obtain an image quality score that is greater than a threshold image quality.

Other examples of inputs to the control system 704 for generating the guidance instructions include physiological data for the patient, such as respiratory/breathing cycle data, ECG data, etc., MRI/CT scan data from a previous MRI or CT scan, and previous ultrasound examination data. Another example of an input to the control system 704 for generating the guidance instructions include protocol data, such as a current step of an ultrasound protocol. Other examples of inputs to the control system 704 for generating the guidance instructions include IMU data, including IMU data for the ultrasound scanner 702 and IMU data from an IMU on the patient. In an example, the control system 704 implements a compensation system to compensate for patient movement. The compensation system can generate the guidance instructions to cancel movement caused by the patient.

The ultrasound scanner 702 receives the guidance instructions from the control system 704 and adjusts a guidance mechanism 706 of the scanner 702 to expose the guidance to the operator, while concealing the guidance from other personnel, including the patient. In some embodiments, the guidance mechanism includes a container 708 that houses a weight 710. The container 708 is in the shape of a toroid, and the weight 710 can include a ball bearing that can move around the toroid. By moving the weight 710 within the container 708 according to the guidance instructions, the ultrasound scanner 702 exposes a gentle nudge to the user of the scanner 702 that is not detectible by other personnel, such as the patient. For instance, if the weight 710 moves in a direction, it is an indication to the user to move/orient the scanner 702 in the direction to improve the imaging quality. That is, the system 700 can directionally modulate the weight 710 of the scanner 702 to indicate to the user how to move/orient the scanner 702, e.g., provide a “gentle nudge”. In some embodiments, the weight 710 includes a battery implemented to provide power to the ultrasound scanner 702. For instance, one rail of the container 710 can be implemented as a positive rail that makes contact with the battery, and another rail of the container 710 can be implemented as a negative rail (e.g., electrical ground) that makes contact with a different portion of the battery.

In some embodiments, the guidance mechanism includes a first surface 712 and a second surface 714 that house (e.g., “sandwich”) a weight 716. Like the weight 710 in the container 708, the system 700 can directionally modulate the weight 716 of the scanner 702 to indicate to the user how to move/orient the scanner 702, e.g., to provide a “gentle nudge”. In some other embodiments, the guidance mechanism includes a pendulum 718 that can move (e.g., sway) to directionally modulate a weight of the scanner 702 to indicate to the user how to move/orient the scanner 702. The pendulum 718 can be encased in a dampening fluid to dampen the weight as it is moved. In some embodiments, the pendulum 718 is in a cone or other enclosure and the impact of the pendulum against the cone is felt as haptic feedback to provide direction information.

Additionally or alternatively, the guidance mechanism can include a viscous material doped with metal particles that can be magnetically and/or electronically moved to accomplish a directional weight modulation of the scanner 702 to indicate the guidance instructions. Additionally or alternatively, the guidance mechanism can include fluid moved with a pump to accomplish a directional weight modulation of the scanner 702 to indicate the guidance instructions. Additionally or alternatively, the guidance mechanism can include a linear actuator to accomplish a directional weight modulation of the scanner 702 to indicate the guidance instructions.

FIG. 8 illustrates some embodiments of an example system 800 with a multi-functional ultrasound scanner 802 in accordance with the present invention. Referring to FIG. 8, the scanner 802 includes a display 804 that can be implemented to display guidance instructions, or visual representations thereof, to indicate how to hold/orient the scanner 802. The guidance instructions can be generated with the control system 704, as previously described. The display 804 can be included at any suitable position of the scanner 802. In some embodiments, the display 804 is included at a proximal end portion (206 in FIG. 2) of the scanner 802, so that the display 804 can be hidden from view of other personnel and/or the patient. Additionally or alternatively, the display 802 can be included in smartphone, tablet, ultrasound machine, AR/VR goggles, AR contact lenses, and the like. The system 800 includes three examples of user interfaces 806, 808, and 810 that can be displayed on the display 804 to indicate the guidance instructions.

In some embodiments, the user interface 806 includes a visual representation 812 that includes a bubble, and to implement the guidance instructions, the user may rotate the scanner 802 about its longitudinal axis (208 in FIG. 2) so that the bubble of the visual representation 812 aligns with the arrowhead 814. The user interface also a visual representation 816 that also includes a bubble. To further implement the guidance instructions, the user may tilt the scanner 802 so that the bubble of the visual representation 816 aligns with the cross-hairs 818.

In some embodiments, the user interface 808 includes visual representations 820 and 822. The visual representation 822 includes a bubble that dynamically moves as the scanner 802 is moved, and/or based on the guidance instructions. For instance, the system 800 can position the bubble of the visual representation 822 within the user interface 808 based on the guidance instructions. The visual representation 820 also includes a bubble. To implement the guidance instructions, the user may move (e.g., rotate, translate, tilt, etc.) the scanner 802 so that the bubble of the visual representation 820 aligns with the bubble of the visual representation 822, e.g., so that the bubble of the visual representation 822 is centered within the bubble of the visual representation 820.

In some embodiments, the user interface 810 includes visual representations 824 and 826. The visual representation 824 includes a straight arrow, and the visual representation 826 includes a bent arrow. To implement the guidance instructions, the user may translate the scanner 802 in a direction of the vector visual representation 824, and rotate the scanner 802 (e.g., about its longitudinal axis 208) in a direction of the vector of the visual representation 826. The vectors of the visual representations 824 and 826 can dynamically change their directions and magnitudes based on the guidance instructions. For example, the magnitude of the vectors can change so that they are proportional to the amount of movement/adjustment needed. The vectors collapse (to zero length) when scanner 802 is correctly oriented/positioned.

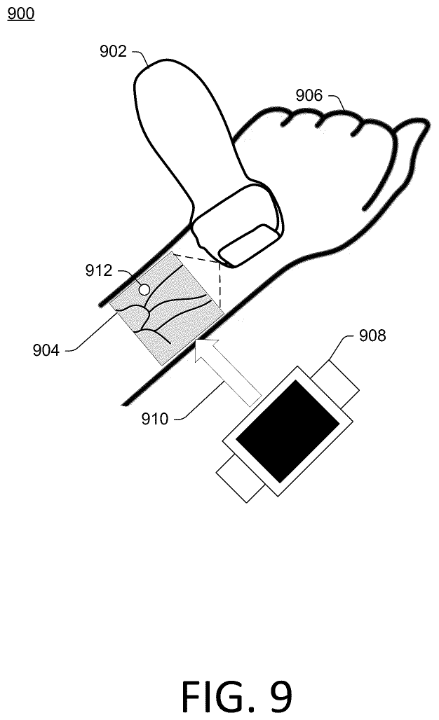

FIG. 9 illustrates some embodiments of an example system 900 with a multi-functional ultrasound scanner 902. Referring to FIG. 9, the multi-functional ultrasound scanner 902 includes an infrared sensor configured to generate an infrared image 904 of a patient anatomy, e.g., a vascular structure. In some embodiments, the multi-functional ultrasound scanner 902 also includes a light source, such as a MEMS projector that can project the infrared image 904 onto the patient's skin. The multi-functional ultrasound scanner 902 can include an IMU and registration system to determine the position of the infrared image 904 relative to the patient anatomy. The multi-functional ultrasound scanner 902 can also include memory to store images acquired by the system. Hence, the multi-functional ultrasound scanner 902 can project the infrared image 904 onto the patient's skin at a position so that the patient anatomy depicted in the infrared image 904 aligns with the patient anatomy underneath it. For example, the vascular architecture depicted in the infrared image 904 in FIG. 9 represents the vascular architecture of the patient 906 at the location where the infrared image 904 is projected.

In some embodiments, the system 900 also includes a wearable ultrasound scanner 908, e.g., a patch-based ultrasound scanner. The scanner 104-2 in FIG. 2 is an example of the wearable ultrasound scanner 908. Because the anatomy in the infrared image 904 is aligned with the patient anatomy, the operator can place (indicated by the arrow 910) the wearable ultrasound scanner 908 onto the patient 906 using the infrared image 904 as a guide for placement of the wearable ultrasound scanner 908.

In some embodiments, the wearable ultrasound scanner 908 is a multi-functional ultrasound scanner that includes an infrared sensor. Once the wearable ultrasound scanner 908 is placed on the patient, it can generate an additional infrared image (not shown in FIG. 9). The system can compare the infrared image 904 captured by the multi-functional ultrasound scanner 902 to the additional infrared image captured by the wearable ultrasound scanner 908. Based on this comparison, e.g., to an extent that the infrared images differ in position relative to one another, the system 900 can make an adjustment to the wearable ultrasound scanner 908, such as to set an ultrasound imaging parameter, beamforming parameter, depth, gain, etc. In some embodiments, the wearable ultrasound scanner 908 includes a movable array that can be mechanically adjusted (e.g., as described in more detail with regards to FIG. 10). Based on the comparison of the infrared images, the system 900 can adjust the position of the ultrasound array of the wearable ultrasound scanner 908. Additionally or alternatively, the wearable ultrasound scanner 908 can generate an ultrasound image once it is placed on the patient, and based on the ultrasound image, the system 900 can adjust the position of the ultrasound array of the wearable ultrasound scanner 908.

In some embodiments, the system determines an insertion point for a needle, e.g., with a machine-learned model, and indicates the insertion point on the infrared image 904. For instance, the infrared image 904 indicates the insertion point 912. In some embodiments, the needle is inserted at the insertion point 912 prior to the placement of the wearable ultrasound scanner 908.

FIG. 10 illustrates some embodiments of an example system 1000 with a multi-functional ultrasound scanner 1002 in accordance with the present invention. Referring to FIG. 10, the multi-functional ultrasound scanner 1002 is an example of the scanner 104, and can therefore include a handheld scanner for external operation (e.g., the scanner 104-1), a wearable ultrasound scanner (e.g., the scanner 104-2), and a scanner configured for internal operation (e.g., the scanner 104-3).

In some embodiments, the scanner 1002 includes a lens 1004 that covers circuitry 1006 of the multi-functional ultrasound scanner 1002. The circuitry 1006 can include one or more ultrasound transducer arrays, one or more infrared sensors, and one or more microphones. In some embodiments, the circuitry 1006 includes at least one of a magnetic sensor, a laser, and a thermometer. The circuitry 1006 is mechanically coupled to an adaptive mount 1008 that can be implemented to move the circuitry 1006 within the scanner 1002. For example, the lens 1004 has dimensions D1×D3, and the circuitry 1006 has dimensions D2×D4, with D1>D2 and D3>D4. Hence, the adaptive mount 1008 can move the circuitry 1006 in directions 1010 and 1012, with the circuitry 1006 remaining positioned so that its sensors (e.g., ultrasound arrays, IR sensor, and microphone) can transmit and receive through the lens 1004. That is, the lens 1004 is wider and longer than the footprint of the circuitry 1006, to accommodate the movement of the circuitry 1006.

In some embodiments, the system 1000 is configured to implement guidance instructions, e.g., generated by the control system 704 as previously described, including to adjust the position of the circuitry 1006 by moving the adaptive mount 1008. In some embodiments, the system can automatically and continuously move the adaptive mount 1008 and adjust the circuitry 1006 during an ultrasound examination, to continuously improve the imaging quality and viewpoint (relative to a conventional system that does not include an adaptive mount) as the user moves the scanner 1002. Additionally or alternatively, the system 1000 can electronically steer one or more sensor arrays (e.g., an ultrasound array, microphone array, IR sensor array, etc.) via beamforming. Additionally or alternatively, the system 1000 includes array movement both in both a linear way and a curved way for the same transducer array.

In some embodiments, the system generates tomographic images (e.g., cross-sectional images of an anatomy). For example, the system can collect image data of the patient anatomy from multiple angles as the adaptive array 1008 is moved across the lens 1004, and process the image data from the multiple angles to generate tomographic images. The tomographic images can include tomographic ultrasound images, tomographic infrared images, and tomographic composite images (e.g., based on ultrasound and infrared data from multiple angles). In some embodiments, the system generates panoramic images (e.g., wide format images) by collecting image data as the adaptive array 1008 is moved across the lens 1004, and stitching together the image data into a wide format, panoramic image. The panoramic images can include panoramic ultrasound images, panoramic infrared images, and panoramic composite images (e.g., based on ultrasound and infrared data from different positions of the adaptive array 1008).

Note that in some embodiments, the techniques described in FIG. 7 and its associated text can be combined with those described in FIG. 10 and its associated text. Similarly, the techniques described in FIG. 8 and its associated text can be combined with those described in FIG. 9 and its associated text.

In some embodiments, audio information captured with the ultrasound data by the multi-functional ultrasound scanner can be used to provide guidance to a user (e.g., clinician). For example, the audio that is captured can enable a user to move the multi-functional ultrasound scanner to peak acoustic energy and may enable the user to hear some bodily function of a patient (e.g., mitral value regurgitation, etc.) that occurs while viewing an image that might be missed because such a bodily function only appears slightly in an ultrasound image.

FIG. 11 illustrates some embodiments of an example system 1100 for processing data from a multi-functional ultrasound scanner. Referring to FIG. 11, for example, the data can be obtained with the multi-functional ultrasound scanner 1002. The system 1100 includes ultrasound image data 1102 captured by the multi-functional ultrasound scanner, and a region 1104 of the ultrasound image data 1102 that is less than the entirety of the ultrasound image data 1102. In an example, the system selects the size of the region 1104 so that it can be displayed on the ultrasound machine 102. In some embodiments, the system 1100 is configured to implement guidance instructions, e.g., generated by the control system 704 as previously described, including to select the region 1104 from the ultrasound image data 1102, and display the region 1104 on the ultrasound machine 102. In some embodiments, the system 1100 can automatically and continuously select and display the region 1104 during an ultrasound examination, to continuously improve the imaging quality and viewpoint.

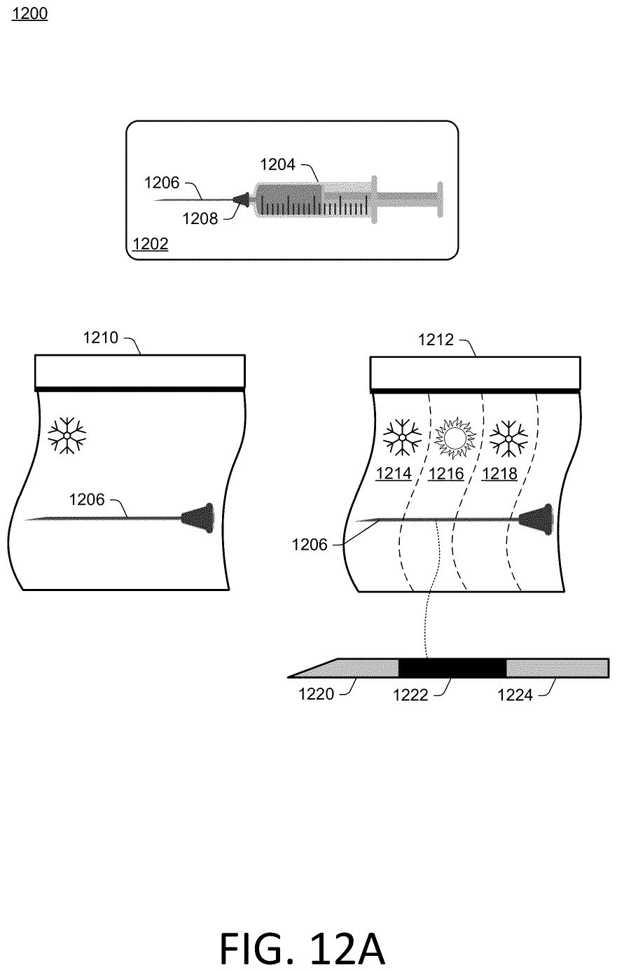

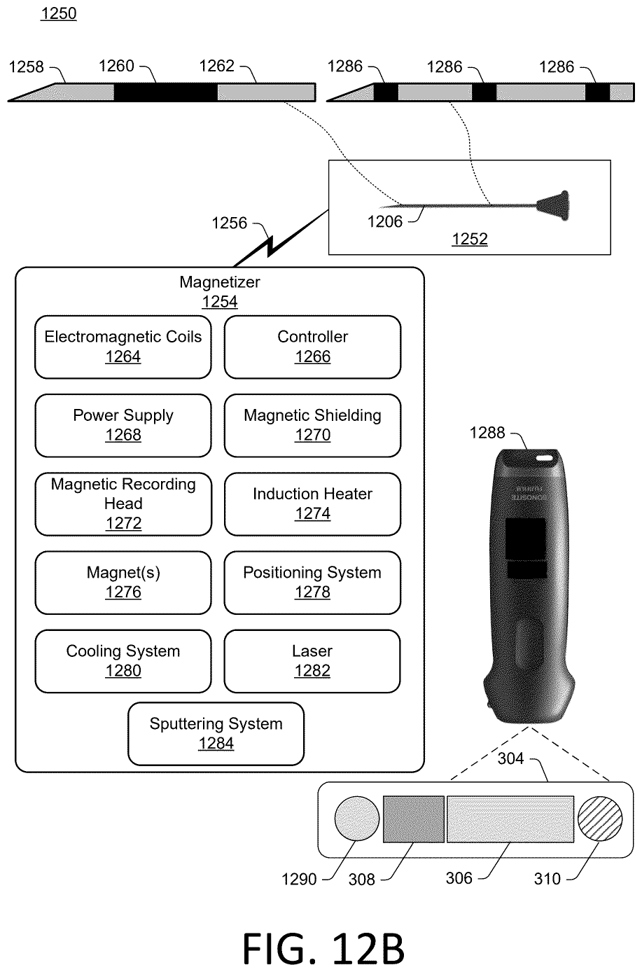

FIG. 12A illustrates some embodiments of example system components 1200 for use with a multi-functional ultrasound scanner in accordance with the present invention. Referring to FIG. 12A, the components 1200 include an interventional instrument 1202 (e.g., a needle) and means to adjust the temperature of the interventional instrument 1202 so that it can be imaged with a multi-functional ultrasound scanner during an ultrasound-guided insertion procedure. Because the temperature of the interventional instrument 1202 can be adjusted, it can be more easily detected with an infrared sensor (e.g., the infrared sensor 308) of the multi-functional ultrasound scanner.

In some embodiments, the interventional instrument 1202 includes a barrel 1204, a needle 1206, and hub (e.g., a luer lock) 1208. In this example, the interventional instrument 1202 comprises a syringe, and the needle 1206 can be inserted into a patient as part of an ultrasound-guided procedure to deliver fluid (e.g., medication), or remove fluid (e.g., blood). In some embodiments, the needle 1206 is cooled prior to the insertion procedure. For instance, the components 1200 include packaging 1210 that houses the needle 1206 in a sterile environment (e.g., the interior of the packaging 1210). The packaging 1210 can include a cooling agent that when activated reduces the temperature of the needle 1206. In some embodiments, the cooling agent is activated by applying force to the packaging 1210 to mix a first component of the cooling agent with a second component of the cooling agent. When mixed, the components of the cooling agent act to cool the needle 1206. For many, if not most, ultrasound-guided needle insertions, the needle 1206 remains cold enough so that the infrared sensor of the multi-functional ultrasound scanner can distinguish its temperature from temperatures of the patient anatomy. Hence, a composite image (e.g., the composite image 408) generated by the system based on the infrared data and ultrasound data can include a depiction of the needle 1206 while inserted that is easily visualized and distinguished from the patient anatomy.

In some embodiments, the needle 1206 can continuously be temperature adjusted (e.g., heated or cooled) during the procedure, while the needle is inserted into the patient. For instance, the components 1200 can include a wire run down the length of the needle 1206, to maintain or continuously adjust its temperature (not shown in FIG. 12A). In some embodiments, the system includes a synchronizer circuit to synchronize the temperature adjustment based on the ultrasound imaging. For example, to avoid smearing or distorting the ultrasound data due to the spreading of heat from the needle 1206, the system can synchronize (e.g., pulse) the heat applied to the needle 1206 so that the heat is applied when the ultrasound imaging is disabled.

In some embodiments, the components 1200 also include packaging 1212 that houses the needle 1206 in a sterile environment (e.g., the interior of the packaging 1212). The packaging 1212 can include multiple sections that can adjust the temperature of the needle 1206 separately. For instance, the section 1214 can set the temperature of the needle 1206 that it overlaps to a first temperature, the section 1216 can set the temperature of the needle 1206 that it overlaps to a second temperature, and the section 1218 can set the temperature of the needle 1206 that it overlaps to a third temperature. In one example, at least one of the first, second, and third temperatures is greater than room temperature (e.g., 70 deg F), and another of the first, second, and third temperatures is less than room temperature. In some embodiments, at least one of the first, second, and third temperatures is greater than body temperature (e.g., 98.6 deg F), and another of the first, second, and third temperatures is less than body temperature. To set the temperatures of the needle 1206 differently, the sections 1214, 1216, and 1218 can use different cooling agents and/or heating agents, or different mixtures of components of a cooling agent and/or different mixtures of components of a heating agent.

Because of the different temperatures in the sections 1214, 1216, and 1218, a temperature pattern is imparted on the needle 1206. In the example in FIG. 12A, the temperature pattern includes three bands 1220, 1222, and 1224 that correspond to the sections 1214, 1216, and 1218, respectively. The system can detect this temperature pattern on the needle 1206 using the multi-functional ultrasound scanner. In some embodiments, the system determines a position of the tip of the needle 1206 based on the temperature pattern it determines on the needle 1206. For instance, the packaging 1212 can register the needle 1206 when it is being temperature adjusted with respect to the sections 1214, 1216, and 1218. This registration information, together with a known length of the needle can be used by the system to determine the location of the tip of the needle 1206 when it is inserted into the patient. The system can label this location in an image it displays, e.g., an ultrasound image or composite image. The system can label the location of the needle tip with a circle, light, etc.

In some embodiments, the hub (e.g., a luer lock) 1208 includes an infrared sensor that can also include an infrared emitter (not shown in FIG. 12A for clarity). The system can use the infrared sensor on the hub 1208 to image the needle 1206, including the tip of the needle 1206.

Additionally or alternatively to adjusting the temperature of the interventional instrument 1202 as described with respect to FIG. 12A, in some embodiments, the system can induce a magnetic field pattern on the interventional instrument 1202. Accordingly, FIG. 12B illustrates some embodiments of example system components 1250 for use with a multi-functional ultrasound scanner in accordance with the present invention. Referring to FIG. 12B, the components 1250 include the needle 1206 of the interventional instrument 1202, and mechanism to induce a magnetic field pattern on the needle 1206 so that it can be detected and imaged with a multi-functional ultrasound scanner that includes one or more magnetic sensors during an ultrasound-guided needle insertion procedure. Although FIG. 12B is described with respect to inducing a magnetic field pattern on the needle 1206, the system can be used to induce a magnetic field pattern on any suitable interventional instrument 1202, such as a clamp, cutter, forceps, etc.

The components 1250 include a fixture 1252 implemented to hold the needle 1206. The fixture 1252 can include any suitable type of mechanism to secure the needle 1206 while the system induces a magnetic field on it, such as a clamp, strap, bonding, vice, etc. The fixture 1252 is coupled to a magnetizer 1254 via a coupling 1256. The coupling 1256 can include any suitable means to couple data, control signals, components of the magnetizer 1254, laser signals, electronic signals, magnetic signals, heating, cooling, etc. between the fixture 1252 and the magnetizer 1254, such as cables, a communication link, fiber optics, and the like.

The magnetizer 1254 can induce a magnetic field pattern onto the needle 1206. For instance, the magnetic field pattern is represented as sections 1258, 1260, and 1262 on the needle 1206 in FIG. 12B. The sections 1258, 1260, and 1262 can have different magnetic field properties from one another that make up the magnetic field pattern induced by the magnetizer 1254. As an example, the sections 1258, 1260, and 1262 can have different magnetic polarities, field strengths, angles (e.g., in different planes radially from the needle 1206), combinations thereof, and the like. By inducing the different magnetic polarities onto the sections 1258, 1260, and 1262, a multi-functional ultrasound scanner having one or more magnetic sensors can detect the needle 1206 during an insertion procedure and include a visual representation of the needle 1206 on an ultrasound image. Using the detected boundaries between sections 1258, 1260, and 1262 and the length of the needle 1206 that is known a priori to the insertion procedure, the system can determine a location of the tip of the needle 1206 during the procedure, and indicate the location of the needle tip on an ultrasound image, such as with a circle, marker, blinking indicator, etc.

The magnetizer 1254 includes electromagnetic coils 1264, a controller 1266, a power supply 1268, magnetic shielding 1270, a magnetic recording head 1272, an induction heater 1274, magnet(s) 1276, a positioning system 1278, a cooling system 1280, a laser 1282, and a sputtering system 1284. The magnetizer can also include a meter (e.g., a Gauss meter) (not shown in FIG. 12B) to test the magnetic field pattern induced on the needle 1206. In some embodiments, if the magnetic field pattern induced on the needle 1206 does not pass quality criteria as determined using the meter (e.g., the field pattern is too weak, the polarity is inverted, etc.), the system removes or changes the magnetic field pattern on the needle 1206, including to change one or more parameters of the magnetizer 1254 (e.g., a voltage or current in the power supply 1268, an amount of the magnetic shielding 1270 used on the needle 1206, etc.) and/or induce another magnetic field pattern on the needle 1206 that does pass the quality criteria.

The magnetizer 1254 can induce a magnetic field pattern on the needle 1206 in any suitable way. In some embodiments, the magnetizer 1254 uses the electromagnetic coils 1264 to induce the magnetic field pattern on the needle 1206. For example, the system can identify and mark the sections 1258, 1260, and 1262 to be magnetized. The system can wind multiple coils around the sections 1258, 1260, and 1262 of the needle 1206. In an example, the sections 1258, 1260, and 1262 each have different numbers of coils (e.g., coil windings) wound around them. The system can use the positioning system 1278 to ensure the coils 1264 are accurately placed over the sections 1258, 1260, and 1262. In some embodiments, the system (or user) places magnetic shielding materials 1270 to isolate one or more of the sections 1258, 1260, and 1262 to prevent unwanted magnetization. For instance, one or more the sections 1258, 1260, and 1262 can remain unmagnetized as part of the magnetic field induced on the needle 1206. The electromagnetic coils 1264 can be connected to the power supply 1268 and controlled by the controller 1266 to sequentially or simultaneously energize the coils 1264 according to the desired magnetic field pattern to be induced on the needle 1206. The properties (e.g., the strength) of the magnetic field pattern can be adjusted by the voltage or current of the power supply 1268, and the duration it is applied.

In some embodiments, the system uses the magnetic recording head 1272 to induce the magnetic field pattern on the needle 1206. For instance, the sections 1258, 1260, and 1262 on the needle 1206 to be magnetized can be identified and located (e.g., registered with respect to the fixture 1252 and the positioning system 1278). The magnetic shielding 1270 can be placed over one or more of the sections 1258, 1260, and 1262 to prevent undesired magnetization as part of the induced magnetic field pattern. The magnetic recording head 1272 can also be registered with the positioning system 1278 so that the magnetic recording head 1272 can be positioned over the sections 1258, 1260, and 1262 (e.g., sequentially placed over each of the sections). The controller 1266 can instruct the magnetic recording head 1272 to move via the positioning system 1278 to the different sections 1258, 1260, and 1262 in any desired sequence, and apply a magnetic field when the magnetic recording head 1272 is over the desired sections. For instance, the controller 1266 can move the magnetic recording head 1272 to each of the sections 1258, 1260, and 1262 and sequentially activate the magnetic field. The magnetic field applied to each of the sections 1258, 1260, and 1262 can have different properties, such as by adjusting the voltage or current of the power supply 1268, and the duration it is applied.

In some embodiments, the system uses the induction heater 1274 to induce the magnetic field pattern on the needle 1206. The induction heater can include one or more focused induction coils. The sections 1258, 1260, and 1262 can be registered in the positioning system 1278, and the controller 1266 can position the induction coils over the sections using the positioning system 1278. The controller 1266 can apply a magnetic field using the magnet(s) 1276, e.g., an electromagnet, during heating and cooling phases. For instance, one or more the sections 1258, 1260, and 1262 can be heated using induction, and cooled with the cooling system 1280 while maintaining the magnetic field.

In some embodiments, the system uses the laser 1282 to induce the magnetic field pattern on the needle 1206. The sections 1258, 1260, and 1262 can be registered in the positioning system 1278, and the controller 1266 can position the laser via the positioning system 1278 over one of the sections 1258, 1260, and 1262. The controller can cause the magnet 1276 to apply a magnetic field during heating with the laser 1282. For instance, the system can coordinate laser 1282 activation with magnetic field application from the magnet(s) 1276 so that the target section is heated with the laser 1282 and cooled with the cooling system 1280 while maintaining the magnetic field.

In some embodiments, the system can sputter a thin layer of magnetic film on the needle 1206 surface. For instance, the magnetic strip pattern 1286 can be applied to the needle 1206 by the sputtering system 1284 included in the magnetizer 1254. To apply the magnetic strip pattern 1286, e.g., a magnetic coating in the pattern 1286, the sputtering system 1284 can use any suitable magnetic (e.g., ferromagnetic) material, such as iron, nickel, cobalt, permalloy (e.g., a nickel-iron alloy), and rare-earth materials (e.g., neodymium). Prior to applying the magnetic material to form the magnetic strip pattern 1286, the needle 1206 can be cleaned, e.g., ultrasonically and with a solvent, such as acetone. To improve adhesion, the needle 1206 can be etched, such as chemically or abrasively. A mask can be applied to the needle to delineate regions of the needle that are to include the magnetic material from regions that are not to include the magnetic material. In the example in FIG. 12B, the regions of the needle 1206 that exclude the magnetic strip pattern 1286 have been masked. The needle 1206 can then be placed in a vacuum chamber of the sputtering system 1284, and an inert gas (e.g., argon) is introduced into the vacuum chamber. The sputtering system 1284 can then create a plasma field with high-energy ions that impinge the magnetic material, causing atoms to eject off the magnetic material, travel through the vacuum, and settle on the needle 1206 to form the magnetic film making up the magnetic strip pattern 1286. The needle 1206 can be rotated during the sputtering process to ensure an even coating. Duration of the process can control the thickness of the magnetic layer. In some embodiments, the needle 1206 is then heat treated and annealed to improve the magnetic field properties of the magnetic strip pattern 1286 by aligning magnetic domains. In some embodiments, the needle 1206 is then polished to increase smoothness and usability of the needle 1206.

The system components 1250 also include a multi-functional scanner 1288 that is an example of the scanner 104, e.g., the scanner 104-1, the scanner 104-2, and/or the scanner 104-3. The scanner 1288 includes the lens 304 at its distal end portion, as well as the ultrasound transducer array 306, the infrared sensor 308, and the microphone 310, as previously described with respect to the scanner 302 in FIG. 3. The scanner 1288 also includes a magnetic sensor 1290 that can detect the magnetic field pattern induced on the needle 1206 by the magnetizer 1254. The magnetic sensor 1290 can include any suitable number of magnets, such as one or more magnets, including an array of magnets.

### An Example Monitoring System

FIG. 13 illustrates some embodiments of an example system 1300 in a monitoring environment with a multi-functional ultrasound scanner. Referring to FIG. 13, the environment includes a patient 1302 who is wearing a multi-functional ultrasound scanner 1304. The multi-functional ultrasound scanner 1304 is an example of the scanner 104-2 in FIG. 2, e.g., a patch-based, wearable ultrasound scanner. The multi-functional ultrasound scanner 1304 can be placed on the patient 402 over a surgery site, e.g., to monitor the anatomy of the site after the procedure. In some embodiments, the multi-functional ultrasound scanner 1304 can be used to monitor an athlete during a game. In some other embodiments, the multi-functional ultrasound scanner 1304 can be used to monitor growth of a patient anatomy, such as bone growth of a child.

In some embodiments, the multi-functional ultrasound scanner 1304 includes circuitry 1306, as is illustrated in profile in FIG. 13. The circuitry 1306 can include any suitable electronics to process and/or display ultrasound data generated by the multi-functional ultrasound scanner 1304, and communicate data to another device, such as the ultrasound machine 102 or any of the computing devices illustrated in FIG. 13. For example, the circuitry 1306 can include the system electronics 216, the memory 110, the ultrasound control subsystem 218, the ultrasound imaging subsystem 220, the processors 106, the transceiver 112, the display device 108, and the like. The circuitry 1306 can communicate over the network 1318, e.g., via Bluetooth™, Wi-Fi™, etc. In some embodiments, the network 1318 includes a network maintained by a care facility, e.g., an intranet. Additionally or alternatively, the network 1318 can include the Internet.

In some embodiments, the multi-functional ultrasound scanner 1304 also includes one or more ultrasound transducer arrays 1308. The transducer arrays 1308 can include any suitable type of transducer array, including one or more transducer arrays that include one or more elements selected from the group consisting of piezoelectric micro-machined ultrasonic transducer (PMUT) array elements, lead zirconate titanate (PZT) array elements, and capacitive micro-machined ultrasonic transducer (CMUT) array elements. In some embodiments, the transducer arrays 1308 include a multi-array transducer that includes one or more transducer arrays having PZT array elements and one or more additional transducer arrays having PMUT array elements. In some other embodiments, transducer arrays 1308 include a multi-array transducer that includes one or more transducer arrays having PZT array elements and one or more additional transducer arrays having CMUT array elements. In still some other embodiments, the transducer arrays 1308 include a multi-array transducer that includes one or more transducer arrays having PMUT array elements and one or more additional transducer arrays having CMUT array elements.

In some embodiments, the multi-functional ultrasound scanner 1304 is a multi-array ultrasound scanner that includes multiple transducer arrays having PZT array elements. In some embodiments, the multi-functional ultrasound scanner 1304 is a multi-array ultrasound scanner that includes multiple transducer arrays having PMUT array elements. In some embodiments, the multi-functional ultrasound scanner 1304 is a multi-array ultrasound scanner that includes multiple transducer arrays having CMUT array elements.

The multi-functional ultrasound scanner 1304 can include a lens (not shown in FIG. 13 for clarity). The lens can be placed over the transducer arrays 1308 and facing the patient. In some embodiments, the multi-functional ultrasound scanner 1304 also includes a coupling agent 1310 that couples ultrasound from the transducer arrays 1308 to the patient anatomy, as well as reflections of the ultrasound from the patient anatomy back to the transducer arrays 1308. The coupling agent 1310 can be encapsulated in a packet (e.g., a gel pack). In some embodiments, the gel pack is affixed to a tegaderm pad or patch. In some embodiments, the packet can be affixed to the transducer arrays 1308 or a lens covering the transducer arrays. The packet can be held in place with pressure, a pocket or recess to hold the packet, a clip or stand to retain the packet, or combinations thereof. In some embodiments, the coupling agent 1310 can lack a bounding container, like a packet, and instead include a gel or paste. The multi-functional ultrasound scanner 1304 can be placed against the patient skin 1312.

In some embodiments, the multi-functional ultrasound scanner 1304 also includes an infrared sensor 1314 and a microphone 1316 positioned next to the transducer arrays 1308 so that they can receive data from the patient anatomy. The infrared sensor 1314 can include any suitable number of sensor elements to detect infrared radiation, e.g., an array of infrared sensor elements. The microphone 1316 can include any suitable number of microphone elements to receive audio data, e.g., an array of microphone elements. In some embodiments, the circuitry 1306 implements one or more beamformers to beamform (e.g., electronically steer) the infrared sensor 1314 and/or the microphone 1316. In one example, the circuitry 1306 beamforms at least one of the infrared sensor 1314 and the microphone 1316 so that they are focused on a same patient anatomy as the transducer arrays 1308.

In some embodiments, the multi-functional ultrasound scanner 1304 can communicate any suitable data, such as the ultrasound images, infrared images, composite images, audio data, etc., over the network 1318 to a computing device coupled to the network 1318, including the computing device 1320 (e.g., a smart phone or tablet, such as a doctor's personal computing device), a device at a nurse station 1322, a medical archiver 1324, a server 1326, and the ultrasound machine 102. By communicating the data to the nurse station 1322, care facility staff can be kept up to date on the health of a patient's recovery from a procedure, and make appropriate scheduling decisions. In some embodiments, the multi-functional ultrasound scanner 1304 is coupled to a quantum computer (not shown in FIG. 13), which can communicate quantum-processed ultrasound data (including ultrasound data combined with infrared and/or audio data) to the multi-functional ultrasound scanner 1304.

The server 1326 can include one or more server devices, such as a server system maintained by a care facility. A clinician may have access to the server 1326 to review a health status report or examination data for a patient. The medical archiver 1324 can maintain patient data, and update the patient's 1302 records with a health status report provided by the multi-functional ultrasound scanner 1304. The medical archiver 1324 can also provide previous patient data for comparison to current patient data. For instance, a clinician or the patient 1302 can compare a previous inference generated with a machine-learned model to a current inference generated by the machine-learned model. In some embodiments, the circuitry 1306 can implement the machine-learned model. Additionally or alternatively, the computing device 1320, the server 1326, or the ultrasound machine 102 can implement the machine-learned model.

In some embodiments, the patient 1302 wears a health tracking device 1328 that can monitor the patient's 1302 movement and health parameters (e.g., motion, steps, heart rate, oxygen level, etc.). The health tracking device 1328 can include an IMU to monitor the patient's motion, and any suitable sensor to determine the health parameters. In some embodiments, the multi-functional ultrasound scanner 1304 includes the health tracking device 1328. For instance, transducer assemblies and the health tracking device 1328 can be integrated onto a common substrate of the multi-functional ultrasound scanner 1304. The system can include a synchronizer circuit (e.g., implemented in the circuitry 1306) to synchronize the data capture from the transducer arrays 1308 relative to the data indicated by the health tracking device 1328. For example, the synchronizer circuit can gate the ultrasound generated and/or received by the multi-functional ultrasound scanner 1304 with the motion data obtained by the health tracking device 1328 so that the ultrasound data is not obtained while the patient is moving, e.g., bending down, so as not to bias the ultrasound data. For instance, if the system is monitoring spinal compression, the system can capture data for a consistent positioning of the spine to prevent comparison of compression data from a prone spinal position with compression data from a bent spinal position, making the comparison more useful.

In some embodiments, the circuitry 1306 includes a power generation circuit that generates power to charge a battery that powers the scanner 1304. For example, the power generation circuit can convert body heat and/or motion of the patient 1302 into an electrical current to charge (e.g., trickle charge) a battery powering the scanner 1304. In some embodiments, the power generation circuit includes a thermoelectric chip (integrated circuit) that harvests body heat. Additionally or alternatively, the power generation circuit can include a quantum-well detector of the infrared sensor 1314, and generate the charging current from infrared radiation emitted as body heat and sensed by the quantum-well detector of the infrared sensor 1314.

### Example Robotic Systems

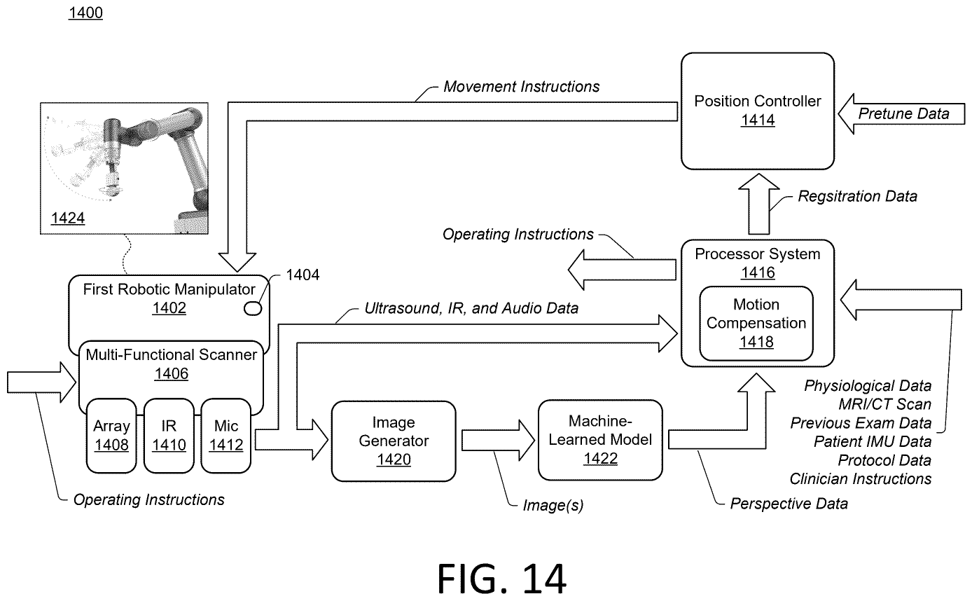

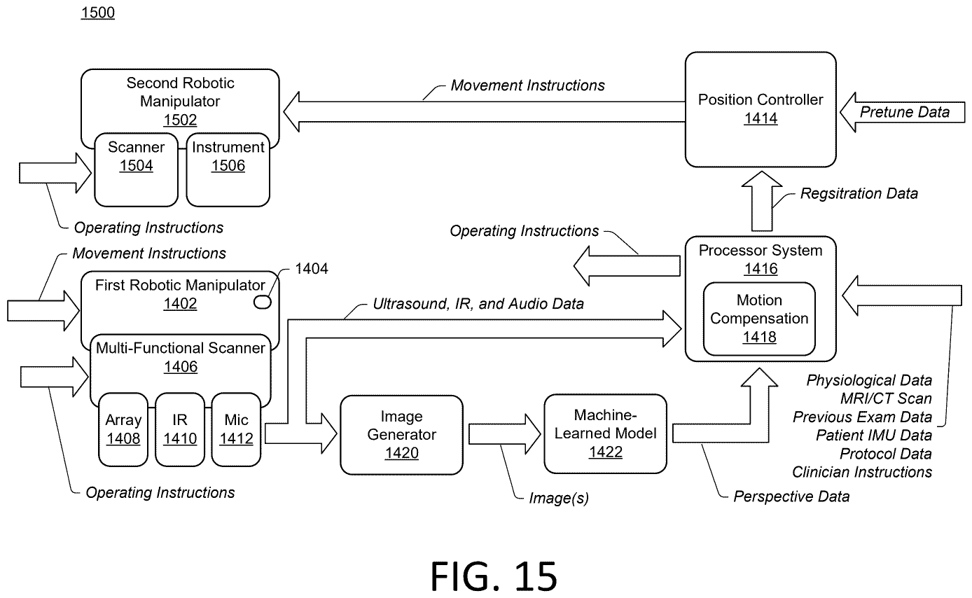

FIGS. 14 and 15 are next described jointly. FIG. 14 illustrates some embodiments of an example robotic system 1400 with a multi-functional ultrasound scanner. Referring to FIG. 14, the system 1400 includes a first robotic manipulator 1402. The first robotic manipulator 1402 is configured to hold a multi-functional ultrasound scanner 1406. FIG. 15 illustrates some embodiments of an example robotic system 1500 with a multi-functional ultrasound scanner in accordance with the present invention. Referring to FIG. 15, the system 1500 includes the first robotic manipulator 1402 and components of the system 1400, as well as a second robotic manipulator 1502 configured to hold a scanner 1504 (e.g., a multi-functional ultrasound scanner such as the scanner 104) and an interventional instrument 1506 (e.g., a needle, such as the syringe 1202).

In some embodiments, the first robotic manipulator 1402 includes an arm configured to hold the multi-functional ultrasound scanner 1406 and that can rotate/articulate about at least one axis. For instance, an example of the first robotic manipulator 1402 is illustrated at the inset 1424. The second robotic manipulator 1502 can also include at least one arm as is illustrated at the inset 1424. In some embodiments, the second robotic manipulator 1502 includes a first arm configured to hold the scanner 1504, and a second arm configured to hold the interventional instrument 1506.

In some embodiments, the first robotic manipulator 1402 includes one or more sensors 1404. For clarity, the sensors 1404 are not illustrated on the second robotic manipulator 1502. In some embodiments, the second robotic manipulator 1502 also includes one or more sensors 1404. The one or more sensors 1404 can include any suitable type of sensor, including a light sensor (a laser, light detection and ranging (LIDAR), etc.), a pressure sensor, a camera, a line scanner, an ultrasound array, etc., and the system 1400 can use sensor data obtained by the one or more sensors 1404 to generate registration data. For instance, the one or more sensors 1404 can read a fiducial marker to generate the registration data.

The multi-functional ultrasound scanner 1406 is an example of the scanner 104, and includes an ultrasound array 1408, an infrared sensor 1410, and a microphone 1412. The ultrasound array 1408 is an example of the array 306; the infrared sensor 1410 is an example of the infrared sensor 308; the microphone 1412 is an example of the microphone 310. As such, the infrared sensor 1410 can include any suitable number of IR sensors configured to receive infrared radiation, and in some embodiments, the infrared sensor 1410 can be electronically steered, e.g., via beamforming. Further, the microphone 1412 can include any suitable number of microphones configured to receive audio, and in some embodiments, the microphone 1412 can be electronically steered, e.g., via beamforming.

In some embodiments, at least one of the infrared sensor 1410 and the microphone 1412 acts as a scout sensor that can obtain information (e.g., infrared data and/or audio data of a patient anatomy), which the system 1400 can use to control the first robotic manipulator 1402 and/or the ultrasound array 1408. For instance, the system 1400 can provide movement instructions to the first robotic manipulator 1402 to move to a location based on the infrared data and/or audio data of a patient anatomy, and provide operating instructions to the ultrasound array 1408 to obtain ultrasound data of a patient anatomy while the first robotic manipulator 1402 is at the location.

In some embodiments, the multi-functional scanner 1406 on the first robotic manipulator 1402 acts as a scout scanner that can obtain information (e.g., ultrasound data, infrared data, audio data), which the system 1500 can use to control the second robotic manipulator 1502, the scanner 1504, and/or the instrument 1506. For instance, the first robotic manipulator 1402 can move the multi-functional scanner 1406 along a patient and follow the first robotic manipulator 1402 with the second robotic manipulator 1502, whose movement is based on data captured by the multi-functional scanner 1406. For example, based on a bone imaged by the IR sensor 1410 of the multi-functional scanner 1406, the system 1500 can provide movement instructions to the second robotic manipulator 1502 and operating instructions (e.g., scan instructions) to the scanner 1504 to image tissue near the bone. In some embodiments, based on ultrasound image data captured by the multi-functional scanner 1406 that depicts a blood vessel, the system can provide (i) movement instructions to the second robotic manipulator 1502 to move to a position proximate to the blood vessel and (ii) instructions to insert a needle of the instrument 1506 into the blood vessel.

In some embodiments, the system 1400, 1500 also includes a position controller 1414, a processor system 1416 that can implement a motion compensation system 1418, an image generator 1420, and a machine-learned model 1422. Ultrasound data (analog-to-digital convert (ADC) samples, beamformed samples, non-scan converted data, scan-converted data, etc.), infrared data, and audio data obtained by the multi-functional ultrasound scanner 1406 that is mounted to the first robotic manipulator 1402, are provided to the image generator 1420 and the processor system 1416. In some embodiments, the image generator 1420 generates one or more images from the ultrasound data, infrared data, and audio data and provides the images to the machine-learned model 1422. The images can include ultrasound images, infrared images, composite images (e.g., 408), and images representing the audio data (e.g., a spectrogram, a time-frequency distribution, a Wigner distribution, and the like). Although not illustrated in FIG. 14 for clarity, the image generator 1420 can additionally or alternatively provide the images to the processor system 1416. In some embodiments, the sensor data from the sensors 1404 is grouped with the ultrasound data, IR data, and/or audio data and provided along with the this data to the processor system 1416 and/or the image generator 1420.

In some embodiments, the machine-learned model 1422 receives the images from the image generator 1420 and generates perspective data that can represent a perspective for the images. For example, the perspective data can include a vector that represents an angle of a viewer or source of the image. The vector can be relative to a coordinate system. In some embodiments, the vector can be relative to a previous image (e.g., a previous ultrasound image). In some embodiments, the perspective data includes data representing an image view or view of anatomy in an image. In some embodiments, the perspective data includes an amount of movement of a perspective from one image to another image. The movement can be caused by a patient's breathing, movement of a transport vehicle (e.g., an ambulance, life flight) transporting the patient, movement of an ultrasound scanner, and the like. The machine-learned model 1422 provides the perspective data to the processor system 1416.

In some embodiments, the processor system 1416 receives the ultrasound data, IR data, and/or audio data from the multi-functional scanner 1406 mounted on the first robotic manipulator 1402, the perspective data from the machine-learned model 1422, and various secondary data, such as physiological data, magnetic resonance imaging (MRI) scan data or computed tomography (CT) scan data, previous ultrasound data, patient IMU data, protocol data, clinician instructions, and the like. The physiological data can include any suitable data representing a physiological process of the patient, such as electrocardiogram (ECG) data, breathing/respiratory data, etc. The MRI/CT scan data can include imaging data for the patient other than ultrasound data, such as imaging data from an MRI scan, CT scan, etc. The previous ultrasound data can include ultrasound data (e.g., ultrasound images) from one or more previous ultrasound examinations for the patient. The patient IMU data can include positional and orientation data from one or more IMUs worn by the patient. The protocol data can include any configuration data for the system pertaining to an ultrasound protocol, such as a current protocol step, a next protocol step, instructions for configuring an ultrasound scanner and/or ultrasound machine for performing a protocol step, etc. The clinician instructions can include user input from a clinician for the system 1400, such as configuration data for the system, movement instructions for one or more of the robotic manipulators (e.g., the first robotic manipulator 1402), etc.

In some embodiments, based on the data received by the processor system 1416, the processor system 1416 generates registration data for one or more components of the system 1400. The registration data can include positional and/or orientation data for any component of the system 1400, 1500, including the first robotic manipulator 1402, the multi-functional scanner 1406, the sensors 1404, an interventional instrument 1506, the robotic manipulator 1502, the patient, etc. The positional and/or orientation data can represent these components in a coordinate system (e.g., 6 DOF in the coordinate system 228). In some embodiments, the processor system 1416 provides the registration data to the position controller 1414. The processor system 1416 also generates operating instructions for the multi-functional scanner 1406. The operating instructions can configure the ultrasound array 1408, an infrared sensor 1410, and a microphone 1412 for operation, including for an imaging mode (B-mode, super harmonic tissue imaging, sub-harmonic tissue imaging, etc.), infrared scanning, audio data capture, beamforming parameters, image combining (e.g., see FIGS. 4 and 5), etc., and instruct the multi-functional scanner 1406 when to obtain data (e.g., at what positions in the coordinate system to obtain the data). In some aspects, the operating instructions can be provided to a second robotic manipulator (e.g., the second robotic manipulator 1502) for operating a medical device such as a needle of the instrument 1506. Example operating instructions for operating the needle can include inserting the needle into the patient to a predefined depth, pushing a plunger of the needle to inject a fluid into the patient, pulling the plunger of the needle to withdraw fluid from the patient, etc.

In some embodiments, the position controller 1414 receives the registration data from the processor system 1416 and generates movement instructions for the robotic manipulators 1402 and 1502. The movement instructions can instruct the robotic manipulators 1402 and 1502 to move to a location in the coordinate system, including an orientation (e.g., 6 DOF). For instance, a robotic manipulator can implement one or more axes of operation (e.g., a robotic manipulator can include multiple joints that couple components of the robotic manipulator that can operate independently with 6 DOF). In some embodiments, the movement instructions can instruct each of the components in each of the axes of operation to move to a particular location with a particular orientation. In this way, a robotic manipulator can place a device mounted to it (e.g., an ultrasound scanner or interventional instrument) at any desired location and with any desired orientation in the coordinate system. In some embodiments, the movement instructions can instruct the second robotic manipulator 1502 to move to a particular location with a particular orientation and insert a needle of the instrument 1506 into the patient to a predefined depth determined based on the registration data. In some embodiments, the movement instructions can instruct the first robotic manipulator 1402 to move to a particular location with a particular orientation for additional scanning of the patient with the multi-functional scanner 1406, such as scanning with a different sensor. For instance, the multi-functional scanner 1406 can generate infrared data with the IR sensor 1410 at a first location, and then move to a second location to generate additional image data (e.g., ultrasound data) with the ultrasound array 1408.

In some embodiments, the movement instructions are based on pre-tune data received by the position controller 1414. The pre-tune data can include any suitable data to instruct a robotic manipulator to move that may not be based on data generated by the ultrasound scanners or that is not part of the secondary data received by the processor system 1416. For instance, the pre-tune data can include initial movement instructions (e.g., positional and/or orientation data) for a robotic manipulator from a previous ultrasound examination, default values for positional and/or orientation data, etc. By using the pre-tune data, the system 1400, 1500 can begin scanning automatically, without explicit positional and/or orientation instructions provided by a clinician or derived from a current use of the system.

In some embodiments, the pre-tune data is based on reading a fiducial marker. For instance, the fiducial marker can include a quick-response (QR) code placed on the patient by a clinician. The QR code can include data to instruct the system 1400, 1500 to use a robotic manipulator and multi-functional scanner, data to instruct the system 1400, 1500 on particular images (ultrasound, infrared, and/or audio) to capture, a protocol to follow, etc. Hence, a clinician can perform a first ultrasound examination and convey instructions to a robotic manipulator via the QR code so that the robotic manipulator can repeat the examination, even in the absence of the clinician.

In some embodiments, the system 1400, 1500 implements a motion compensation system 1418 that can position and orient a robotic manipulator (e.g., the second robotic manipulator 1502) to dynamically adjust for a motion. For instance, the motion can be due to a patient's movement, such as due to breathing. Hence, the system can include a respiratory sensor to indicate a patient's breathing cycle. Additionally or alternatively, the multi-functional scanner 1406 can generate data for multiple images over one or more cycles of the patient's breathing cycle to create a history of images (e.g., a set of ultrasound, infrared, and/or audio images). In some embodiments, the motion compensation system 1418 can determine positional differences of the patient anatomy over a set of images generated over a duration of time, such as one or more breathing cycles of the patient. In some embodiments, using this history of images, the processor system 1416 can predict a next position of the patient anatomy relative to a current position and include an offset in the registration data representing this prediction, and then, the position controller 1414 can generate the movement instructions for the second robotic manipulator 1502 so that ultrasound data captured by the scanner 1504 is from a position to compensate for the motion. In this way, the system can generate consistent and stable images that reduce artifacts caused by motion. By reducing the artifacts caused by motion, a clinician can, for example, during a needle procedure, be confident that a needle tip is being inserted at a desired location and at a time of the breathing cycle that is not affecting the images generated by the system. Other sources of motion for which the system 1400, 1500 can compensate in this way include motion caused by transport, such as in a helicopter or ambulance.

### An Example User Interface

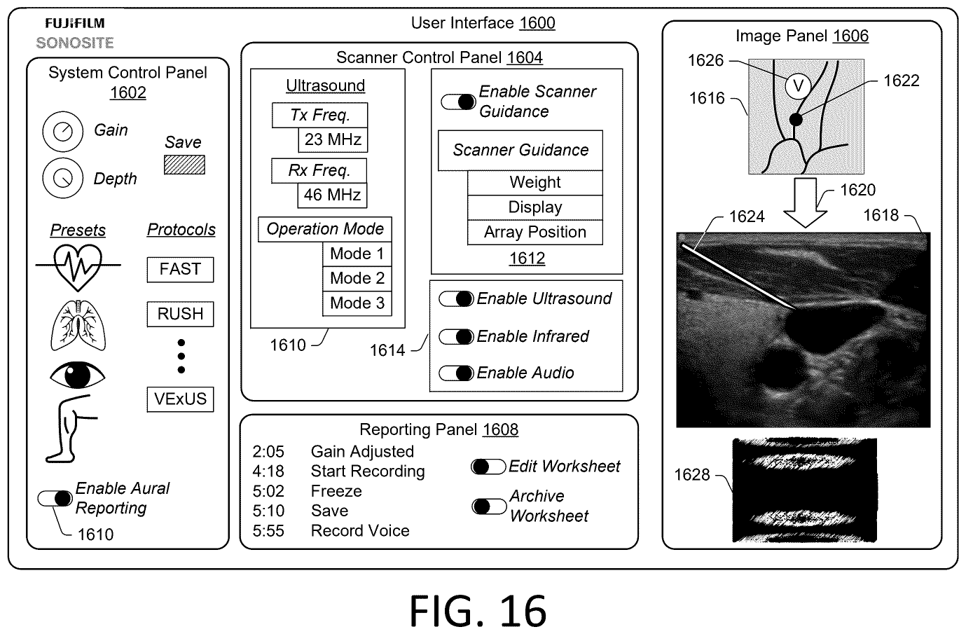

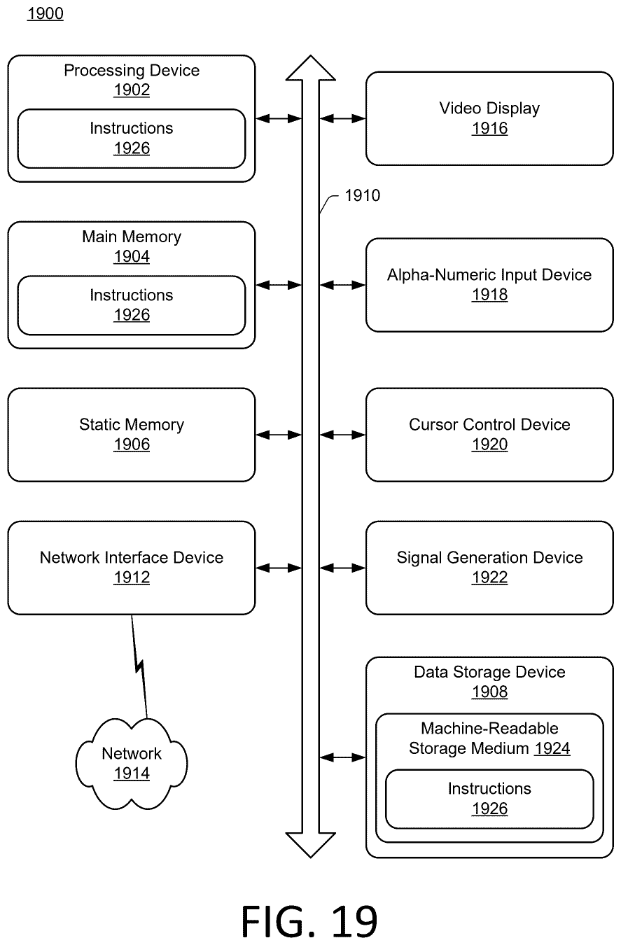

FIG. 16 illustrates some embodiments of an example user interface 1600 of a system with a multi-functional ultrasound scanner. Referring to FIG. 16, the user interface 1600 includes a system control panel 1602, a scanner control panel 1604, an image panel 1606, and a reporting panel 1608. The user interface 1600 can be displayed on any suitable device, such as the ultrasound machine 102, the ultrasound scanner 104, the computing device 1900 in FIG. 19, any computing device illustrated in FIG. 13, etc.

The system control panel 1602 can include any suitable controls for configuring an ultrasound system with a multi-functional ultrasound scanner. In the example in FIG. 16, the system control panel 1602 includes ultrasound controls for adjusting gain and depth, saving an image, and selecting examination presets. The examination presets are represented by selectable icons for a cardiac examination, a respiratory examination, an ocular examination, and a muscular-skeletal examination. These examination presets, when selected, can configure the ultrasound machine with predetermined values of gain and depth, and other imaging parameters (e.g., beamformer settings, filter coefficients, amplitude settings, etc.). The system control panel 1602 also includes options to select a protocol, including a Focused Assessment with Sonography for Trauma (FAST) protocol, a Rapid Ultrasound for Shock and Hypotension (RUSH) protocol, and a Venous Congestion Evaluation using Ultrasound (VExUS) protocol. A user can select one of these example protocols, and in response, the system can configure itself for an examination in accordance with the protocol, including to display a protocol panel in the user interface 1600 (not shown for clarity) with guided steps needed to complete the selected protocol. In some embodiments, The system control panel 1602 also includes a selection (e.g., an electronic rocker switch) to enable aural reporting in accordance with the present invention, such as can be implemented with tactile gestures (e.g., taps and swipes) that can be detected by a microphone on a multi-functional scanner as is described with regards to FIG. 6. Responsive to the selection to enable aural reporting (e.g., via the rocker switch in the system control panel 1602), the user interface 1600 displays the reporting panel 1608.

In some embodiments, the user interface 1600 also includes the scanner control panel 1604 that includes controls 1610 for configuring one or more arrays of an ultrasound transducer assembly of a multi-functional scanner. The controls 1610 includes options to select transmit and receive frequencies for one or more ultrasound arrays of a multi-functional scanner. In the example in FIG. 16, a transmit frequency is set via a drop-down menu to 23 MHz, and a receive frequency is set via a drop-down menu to 46 MHz. The controls 1610 also includes a drop-down menu to enable the multi-functional ultrasound scanner according to an operation mode. Example operation modes are described in this example include first, second, and third operation modes. Examples of these operation modes include a high-frequency linear array mode, a wide-view linear array mode, a low-frequency phased array mode, and a transmit/receive linear array mode. Additionally or alternatively, operation modes can include a mode which can enable at least one array of an ultrasound scanner as a linear array and at least one additional array of the ultrasound scanner as a phased array, a mode which can enable the arrays for broadband tissue harmonic imaging (broadband THI), and a mode which can enable the arrays for full-aperture broadband THI operation.

In some embodiments, the scanner control panel 1604 also includes controls 1612 to enable guidance and/or assistance for a user of a system with a multi-functional ultrasound scanner. The control 1612 include a switch to enable scanner guidance, such as is previously described with respect to FIGS. 7, 8, and 10. For instance, the controls 1612 include a drop-down menu with a selection to enable scanner guidance via a weight selection, e.g., to directionally modulate the weight of the multi-functional ultrasound scanner as described with respect to FIG. 7. In some embodiments, the drop-down menu also includes a selection to enable scanner guidance via a display, e.g., with a display of the multi-functional ultrasound scanner as described with respect to FIG. 8. In some embodiments, the drop-down menu also includes a selection to enable scanner guidance via an array position, e.g., with an adaptive mount of the multi-functional ultrasound scanner as described with respect to FIG. 10.

In some embodiments, the scanner control panel 1604 also includes controls 1614 to enable sensors of a multi-functional ultrasound scanner. For example, the controls 1614 include a first switch to enable ultrasound operation, such as by activating the ultrasound array 306 of the multi-functional ultrasound scanner 302. The controls 1614 also include a second switch to enable infrared operation, such as by activating the infrared sensor 308 of the multi-functional ultrasound scanner 302. The controls 1614 also include a third switch to enable audio operation, such as by activating the microphone 310 of the multi-functional ultrasound scanner 302.

In some embodiments, the user interface 1600 also includes the image panel 1606 for displaying any suitable type and number of images. In the example in FIG. 16, the image panel 1606 displays an infrared image 1616 and an ultrasound image 1618 (e.g., a B-mode image). The infrared image 1616 and the ultrasound image 1618 in this example depict the same patient anatomy, e.g., a vascular architecture, in different imaging planes. The image panel 1606 can be implemented to first display the infrared image 1616, and then display the ultrasound image 1618. In some embodiments, the image panel 1606 changes the display from the infrared image 1616 to the ultrasound image 1618, indicated by the arrow 1620. For instance, the image panel 1606 can display the infrared image 1616 until a needle reaches a threshold distance from a patient anatomy (e.g., a vein in the vascular architecture). In some embodiments, when the needle reaches the threshold distance to the patient anatomy, the image panel 1606 can cease display of the infrared image 1616 and begin display of the ultrasound image 1618. Hence, a user can have a continuous view of the patient anatomy and needle from the time the needle is outside the patient until the time the needle reaches the desired patient anatomy. The needle 1624 is depicted in the ultrasound image 1618, and the needle tip 1622 is depicted in the infrared image 1616.

In some embodiments, the image panel 1606 simultaneously displays the infrared image 1616 and the ultrasound image 1618. Additionally or alternatively, the image panel 1606 can display a composite image (e.g., the composite image 408). Additionally or alternatively, the image panel 1606 can display an audio image (e.g., a visual representation of an audio signal), such as the spectrogram 1628 which indicates frequency content of the audio signal vertically and time horizontally. In some embodiments, the infrared image 1616, the ultrasound image 1618, and the spectrogram 1628 indicate the same anatomy. For instance, an ultrasound array, IR sensor, and microphone can all be focused at the same patient anatomy and receive ultrasound, infrared radiation, and audio from the patient anatomy, and the same time (concurrently).

In some embodiments of a multi-functional ultrasound scanner, the system can process one of the infrared image 1616 and the ultrasound image 1618, such as with a machine-learned model, to generate an inference, and label the other of the infrared image 1616 and the ultrasound image 1618 with an indication of the inference. For example, in FIG. 16, the system implements a machine-learned model to process the ultrasound image 1618 with a machine-learned model to generate a classification of a blood vessel as a vein or artery. The system can then label the blood vessel in the infrared image 1616 with the label 1626 to indicate the classification (e.g., “V” for vein).

In some embodiments, the user interface 1600 also includes the reporting panel 1608 for assisted reporting/workflow during an ultrasound examination. In some embodiments, the user interface 1600 can display the reporting panel 1608 responsive to a selection to enable aural reporting (e.g., via the rocker switch in the system control panel 1602). In some embodiments, a user can perform tactile gestures that can be detected by one or more microphones on a multi-functional ultrasound scanner, as previously described with regards to FIG. 6. For instance, a user can use tactile gestures on the multi-functional ultrasound scanner to set an imaging parameter, adjust gain, adjust depth, freeze an image, save an image, begin recording voice spoken into a microphone of the multi-functional ultrasound scanner, etc. In some embodiments, the reporting panel 1608 can illustrate these operations with timestamps of the ultrasound examination. In the example in FIG. 16, the user adjusted the gain at time 2:05, started recording at 4:18, etc. In some embodiments, the reporting panel 1608 includes a control (e.g., a switch) to enable worksheet editing. When this control is enabled, the system can automatically populate a medical worksheet with data indicated in the reporting panel 1608 from the tactile gestures, and display the medical worksheet as it is automatically populated (not shown in FIG. 16 for clarity). Hence, the system can provide reporting assistance by guiding the user through medical worksheet completion. In some embodiments, the reporting panel 1608 includes a control to archive the medical worksheet, e.g., to save it to a medical archiver, VNA, etc. when the user has completed populating and editing the medical worksheet.

### An Example Machine-Learned Model

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form:

f(ŝ,θ)=\hat{y} Equation (1)

In Equation (1), the operator f represents the processing of the machine-learned model based on an input and providing an output. The term ŝ represents a model input, such as ultrasound data. The model analyzes/processes the input ŝ using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

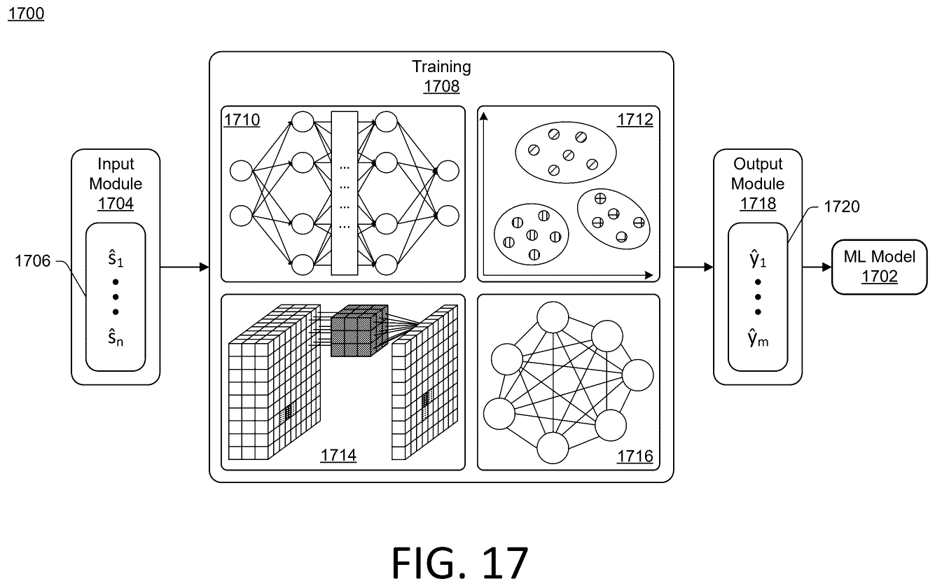

FIG. 17 represents some embodiments of an example machine-learning architecture 1700 used to train a machine-learned model M 1702. Referring to FIG. 17, an input module 1704 accepts an input ŝ 1706, which can be an array with members ŝ1 through ŝn. The input ŝ 1706 is fed into a training module 1708, which processes the input ŝ 1706 based on the machine-learning architecture 1700. For example, if the machine-learning architecture 1700 uses a multilayer perceptron (MLP) model 1710, the training module 1708 applies weights and biases to the input ŝ 1706 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 1710 is described here as an example, any suitable machine-learning technique can be employed, some examples of which include but are not limited to k-means clustering 1712, convolutional neural networks (CNN) 1714, a Boltzmann machine 1716, Gaussian mixture models (GMM), and long short-term memory (LSTM). The training module 1708 provides an input to an output module 1718. The output module 1718 analyzes the input from the training module 1708 and provides an output in the form of ŷ 1720, which can be an array with members ŷ1 through ŷm. The output 1720 can represent a known correlation with the input ŝ 1706, such as, for example, object identification, segmentation, and/or classification.

In some embodiments, the input ŝ 1706 can be a training input labeled with known output correlation values, and these known values can be used to optimize the output ŷ 1720 in training against the optimization/loss function. In some other embodiments, the machine-learning architecture 1700 can categorize the output ŷ 1720 values without being given known correlation values to the inputs ŝ 1706. In some embodiments, the machine-learning architecture 1700 can be a combination of machine-learning architectures. By way of example, a first network can use the input ŝ 1706 and provide the output ŷ 1720 as an input SML to a second machine-learned architecture, with the second machine-learned architecture providing a final output ŷf. In some other embodiments, one or more machine-learning architectures can be implemented at various points throughout the training module 1708.

In some embodiments of machine-learned models, all layers of the model are fully connected. For example, all perceptrons in an MLP model act on every member of ŝ. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

FIG. 18 represents an example model 1800 using a CNN to process an input image 1802, which includes representations of objects that can be identified via object recognition, such as people or cars (or an anatomy, as described in relation to FIGS. 1-17). Referring to FIG. 18, convolution A 1804 can be performed to create a first set of feature maps (e.g., feature maps A 1806). A feature map can be a mapping of aspects of the input image 1802 given by a filter element of the CNN. This process can be repeated using feature maps A 1806 to generate further feature maps B 1808, feature maps C 1810, and feature maps D 1812 using convolution B 1814, convolution C 1816, and convolution D 1818, respectively. In some embodiments, the feature maps D 1812 become an input for fully connected network layers 1820. In this way, the machine-learned model can be trained to recognize certain elements of the image, such as people, cars, or a particular patient anatomy, and provide an output 1822 that, for example, identifies the recognized elements. In some embodiments, an inference generated with an ultrasound system can be appended to a feature map (e.g., feature map B 1808) generated by a neural network (e.g., CNN). In this way, the feature vector and/or inference can be used as a secondary/conditional input to the neural network.

Although the example of FIG. 18 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art can also be employed.

### An Example Device

FIG. 19 illustrates a block diagram of an example computing device 1900 that can perform one or more of the operations described herein, in accordance with some embodiments for a multi-functional scanner. The computing device 1900 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some embodiments, the computing device 1900 is one or more of an ultrasound machine, an ultrasound scanner, an access point, a charging station, and a medical archiver.

The example computing device 1900 can include a processing device 1902 (e.g., a general-purpose processor, a programmable logic device (PLD), etc.), a main memory 1904 (e.g., synchronous dynamic random-access memory (DRAM), read-only memory (ROM), etc.), and a static memory 1906 (e.g., flash memory, a data storage device 1908, etc.), which can communicate with each other via a bus 1910. The processing device 1902 can be provided by one or more general-purpose processing devices such as a microprocessor, a central processing unit, or the like. In an illustrative example, the processing device 1902 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1902 can also comprise one or more special-purpose processing devices such as an application-specific integrated circuit (ASIC), a field programmable gate array (FPGA), a digital signal processor (DSP), a network processor, or the like. The processing device 1902 can be configured to execute the operations described herein, in accordance with one or more aspects of the present disclosure, for performing the operations and steps discussed herein.

The computing device 1900 can further include a network interface device 1912, which can communicate with a network 1914. The computing device 1900 also can include a video display unit 1916 (e.g., a liquid crystal display (LCD), an organic light-emitting diode (OLED), a cathode ray tube (CRT), etc.), an alphanumeric input device 1918 (e.g., a keyboard), a cursor control device 1920 (e.g., a mouse), and an acoustic signal generation device 1922 (e.g., a speaker, a microphone, etc.). In one embodiment, the video display unit 1916, the alphanumeric input device 1918, and the cursor control device 1920 can be combined into a single component or device (e.g., an LCD touch screen).

The data storage device 1908 can include a computer-readable storage medium 1924 on which can be stored one or more sets of instructions 1926 (e.g., instructions for carrying out the operations described herein, in accordance with one or more aspects of the present disclosure). The instructions 1926 can also reside, completely or at least partially, within the main memory 1904 and/or within the processing device 1902 during execution thereof by the computing device 1900, where the main memory 1904 and the processing device 1902 also constitute computer-readable media. The instructions can further be transmitted or received over the network 1914 via the network interface device 1912.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some embodiments, the modules described herein are embodied in the data storage device 1908 of the computing device 1900 as executable instructions or code. Although represented as software implementations, the described modules can be implemented as any form of a control application, software application, signal processing and control module, hardware, or firmware installed on the computing device 1900.

While the computer-readable storage medium 1924 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Procedures

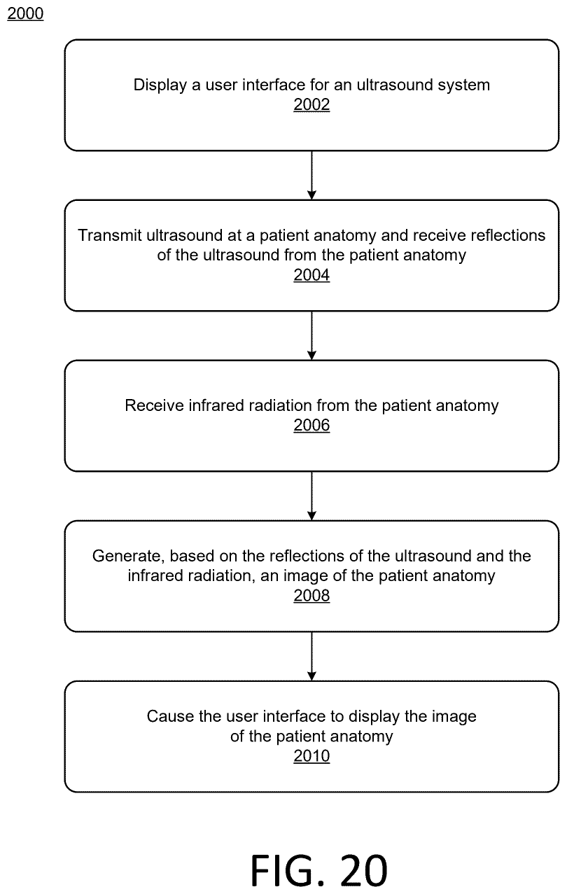

FIG. 20 illustrates some embodiments of a first example method 2000 that can be implemented by an ultrasound system (e.g., the ultrasound system in the environment 100, the ultrasound system in the implementation 200, the ultrasound system 300, the system 400, 500, 600, 1200, 1400, and 1500) for implementing a multi-functional ultrasound scanner. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 19.

Referring to FIG. 20, a user interface for an ultrasound system is displayed (block 2002). Ultrasound is transmitted at a patient anatomy and reflections of the ultrasound are received from the patient anatomy (block 2004). Infrared radiation from the patient anatomy is received (block 2006). Based on the reflections of the ultrasound and the infrared radiation, an image of the patient anatomy is generated (block 2008). A user interface is caused to display the image of the patient anatomy (block 2010).

In some embodiments, the ultrasound scanner includes the infrared sensor. Further, the ultrasound scanner can include a microphone implemented to receive audio from the patient anatomy, and the processor system can implement a machine-learned model that generates, based on the reflections of the ultrasound and the audio, an inference for the patient anatomy. The inference can include at least one of a label, a classification, a segmentation, and an additional image (e.g., a segmentation image). The machine-learned model can be implemented to generate the inference based on the image of the patient anatomy.

In some embodiments, the processor system is implemented to generate a visual representation of the audio and cause the user interface to display the visual representation. Additionally or alternatively, the ultrasound scanner can communicate the audio to an operator of the ultrasound system while the at least one transducer array transmits the ultrasound and receives the reflections. Additionally or alternatively, the processor system can generate, based on at least one of the reflections of the ultrasound, the infrared radiation, the audio, the image, and the inference, guidance instructions. The ultrasound scanner can indicate to an operator of the ultrasound scanner an orientation of the ultrasound scanner based on the guidance instructions. The ultrasound scanner can indicate the orientation such that the indication is imperceptible to the patient. For instance, the indication can include to adjust a position of a weight of the inside the ultrasound scanner, and the scanner can adjust the position of the weight in a manner so that the patient does not observe the adjustment. The adjustment can be noiseless. In some embodiments, the weight includes a battery implemented to provide power to the ultrasound scanner. In an example, the ultrasound scanner includes an additional display device implemented to display a visual representation of the indication of the orientation, as is illustrated with respect to FIG. 10.

In some embodiments, the at least one transducer array includes a piezoelectric micro-machined ultrasonic transducer (PMUT) array, and the PMUT array includes the microphone. For instance, the PMUT array can be manufactured with a microphone on the array. In some embodiments, the system configured the at least one transducer array as a microphone to detect audio signals, e.g., audio signals in the human-perceptible range (e.g., 20 -20 KHz). In some embodiments, the microphone includes a vibrating element to detect the audio. The vibrating element can include a region of an enclosure of the ultrasound scanner. That is, the region of the enclosure of the ultrasound scanner is part of the microphone. In some embodiments, the system includes a disposable microphone that attaches (e.g., magnetically) to the enclosure of the multi-functional ultrasound scanner. For instance, the disposable microphone can be attached to the enclosure of the scanner for a procedure, automatically connect to circuitry within the scanner, and then be discarded after the procedure.

In some embodiments, an ultrasound machine includes a speaker, and the ultrasound scanner includes a microphone. The ultrasound machine can broadcast audio including operation instructions from the speaker to the microphone of the ultrasound scanner, and the at least one transducer array can transmit the ultrasound and receive the reflections based on the operation instructions. The audio can occupy a bandwidth outside a human-perceptible bandwidth, such as 28-32 KHz.

In some embodiments, the ultrasound scanner includes a microphone implemented to detect audio caused by tactile gestures on the ultrasound scanner. The processor system can be implemented to edit a medical worksheet based on the audio caused by the tactile gestures. Additionally or alternatively, the processor system can configure, based on the audio caused by the tactile gestures, at least one transducer array to transmit the ultrasound and receive the reflections.

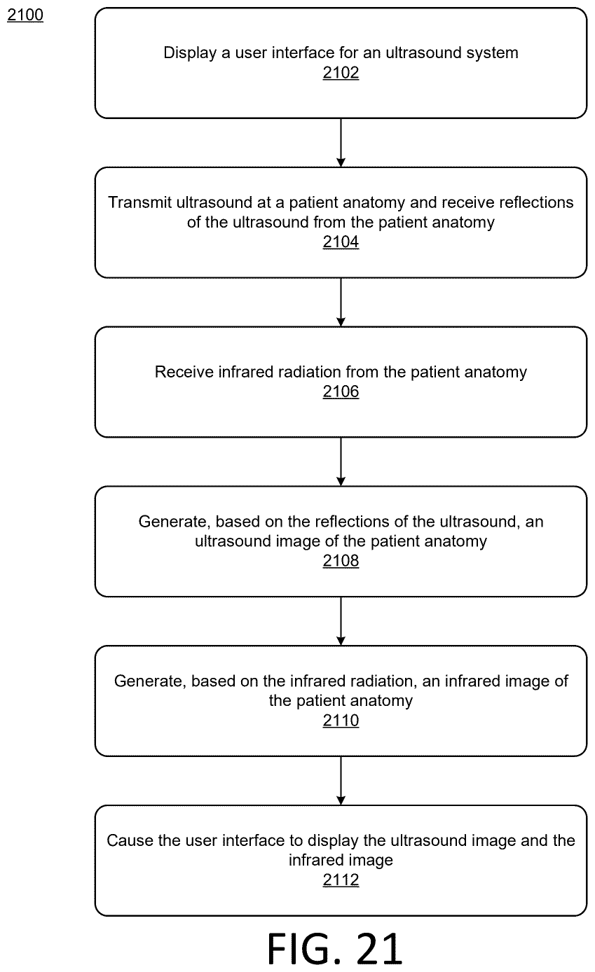

FIG. 21 illustrates some embodiments of a second example method 2100 that can be implemented by an ultrasound system (e.g., the ultrasound system in the environment 100, the ultrasound system in the implementation 200, the ultrasound system 300, the system 400, 500, 600, 1200, 1400, and 1500) for implementing a multi-functional ultrasound scanner. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 19.

Referring to FIG. 21, a user interface for an ultrasound system is displayed (block 2102). Ultrasound is transmitted at a patient anatomy and reflections of the ultrasound are received from the patient anatomy (block 2104). Infrared radiation from the patient anatomy is received (block 2106). Based on the reflections of the ultrasound, an ultrasound image of the patient anatomy is generated (block 2108). Based on the infrared radiation, an infrared image of the patient anatomy is generated (block 2110). The user interface is caused to display the ultrasound image and the infrared image (block 2112).

In some embodiments, a processor system is implemented to cause the user interface to display the infrared image prior to the display of the ultrasound image. Additionally or alternatively, the processor system can cause the user interface to display the infrared image and the ultrasound image simultaneously. In some embodiments, the processor system can generate, based on the ultrasound image and the infrared image, a combination image, and cause the user interface to display the combination image. The combination image can includes a three-dimensional (3D) image of the patient anatomy.

In some embodiments, the processor system can detect, based on at least one of the ultrasound image and the infrared image, an interventional instrument, and determine a position of the interventional instrument relative to the patient anatomy. The processor system can cause, based on the position of the interventional instrument, the user interface to cease the display of the infrared image and start the display of the ultrasound image. In some embodiments, the interventional instrument has a heat pattern applied to it prior to insertion into the patient, and the processor system can detect the interventional instrument in the infrared image and determine the position based on the heat pattern.

In some embodiments, the infrared image includes an interventional instrument having a first temperature that is different from a second temperature of the patient anatomy. The ultrasound system can include a temperature adjustment device implemented to set the interventional instrument to the first temperature prior to insertion of the interventional instrument into the patient. For instance, see FIG. 13. In some embodiments, the temperature adjustment device is implemented to maintain the interventional instrument at the first temperature during the insertion.

In some embodiments, the ultrasound scanner includes a microphone implemented to receive audio from the patient anatomy, and the processor system is implemented to generate an inference of the patient anatomy based on the audio and the ultrasound image. In some embodiments, the processor system can generate a visual representation of the audio and cause the user interface to display the visual representation. Additionally or alternatively, the processor system can classify the patient anatomy based on the audio.

In some embodiments, the ultrasound scanner includes a microphone implemented to receive audio from the patient anatomy. In some embodiments, the processor system can generate, based on the reflections of the ultrasound, additional audio, combine the audio and the additional audio to form combined audio, and communicate the combined audio to an operator of the ultrasound system.

In some embodiments, the processor system can generate, based on the ultrasound image, a label for the patient anatomy. The processor system can cause the label to be applied to the patient anatomy in the infrared image.

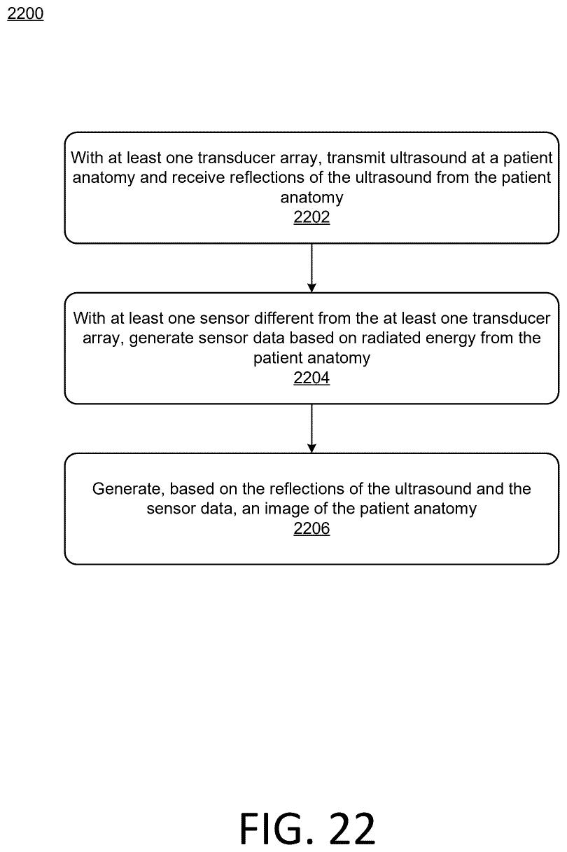

FIG. 22 illustrates some examples of a third example method 2200 that can be implemented by an ultrasound system (e.g., the ultrasound system in the environment 100, the ultrasound system in the implementation 200, the ultrasound system 300, the system 400, 500, 600, 1200, 1400, and 1500) in accordance with the present invention for implementing a multi-functional ultrasound scanner. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 19.

Referring to FIG. 22, with at least one transducer array, ultrasound is transmitted at a patient anatomy and reflections of the ultrasound from the patient anatomy are received (block 2202). With at least one sensor different from the at least one transducer array, sensor data is generated based on radiated energy from the patient anatomy (block 2204). Based on the reflections of the ultrasound and the sensor data, an image of the patient anatomy is generated (block 2206).

In some embodiments, the at least one sensor includes an infrared sensor, the radiated energy includes infrared radiation, and the sensor data includes heat data. Additionally or alternatively, the at least one sensor can includes a microphone, the radiated energy can include sound radiation, and the sensor data can include audio data. Additionally or alternatively, one or more of these sensors can include an infrared sensor and a microphone, the radiated energy can include infrared radiation and sound radiation, and the sensor data can include heat data and audio data. Additionally or alternatively, one or more of these sensors can include a magnetic sensor, the radiated energy can include magnetic radiation, and the sensor data can include magnetic field data.

In some embodiments, the ultrasound scanner is a wearable ultrasound scanner implemented to be patient worn. The processor system can communicate the image of the patient anatomy over a network to a medical archiver. Additionally or alternatively, the processor system can communicate the image of the patient anatomy over a network to a clinician.

Systems, devices, and methods described herein constitute one or more advantages over conventional systems, devices, and methods that do not include a multi-functional ultrasound scanner as disclosed herein, including:
1) Provides better needle visualization than conventional ultrasound systems. By generating a combined image from IR and ultrasound images, needle can be easily detected and tracked as it is at a different temperature than the patient anatomy;
2) Provides real-time solution for accurate placement of wearable ultrasound systems (e.g., patches);
3) Multi-modal data (e.g., IR and ultrasound data) can provide better robotic control than ultrasound data alone;
4) Patients can receive better care with the disclosed systems compared to conventional systems;
5) Enables simultaneous capture of ultrasound image data and stethoscope audio data;
6) Enables better generation of inferences from machine-learned models by exploitation of hidden correlations between image and audio data;
7) Removes subjective interpretations of audio (stethoscope) data;
8) Obsoletes the traditional stethoscope and replaces it with the ultrasound probe;
9) Provides a wireless communication link between the ultrasound machine and probe that does not radiate EM energy. Makes wireless probes feasible underwater and reduces the likelihood of detection by enemies on the battlefield;
10) Provides personal guidance and/or assistance to the clinician holding the probe in a manner that is unobservable to other personnel in the room;
11) Clinicians are more likely to use the guidance/assistance mechanisms than those in conventional systems; and
12) Enables experienced clinicians and less-experienced users (e.g., nurses) to utilize the ultrasound system to its fullest capabilities, and capture optimal ultrasound images.
There are a number of example embodiments described herein.
Example 1 is an ultrasound system having a display device configured to display a user interface for the ultrasound system, an ultrasound scanner having at least one transducer array configured to transmit ultrasound at a patient anatomy and receive reflections of the ultrasound from the patient anatomy, and an infrared sensor configured to receive infrared radiation from the patient anatomy. The ultrasound system also includes a processor system configured to generate, based on the reflections of the ultrasound and the infrared radiation, an image of the patient anatomy and cause the user interface to display the image of the patient anatomy.

Example 2 is the ultrasound system of example 1 that may optionally include that the ultrasound scanner includes the infrared sensor.

Example 3 is the ultrasound system of example 2 that may optionally include that the ultrasound scanner includes a microphone implemented to receive audio from the patient anatomy, and the processor system is configured to implement a machine-learned model that generates, based on the reflections of the ultrasound and the audio, an inference for the patient anatomy.

Example 4 is the ultrasound system of example 3 that may optionally include that the inference includes at least one of a label, a classification, a segmentation, and an additional image.

Example 5 is the ultrasound system of example 3 that may optionally include that the machine-learned model is implemented to generate the inference based on the image of the patient anatomy.

Example 6 is the ultrasound system of example 3 that may optionally include that the processor system is implemented to generate a visual representation of the audio and cause the user interface to display the visual representation.

Example 7 is the ultrasound system of example 3 that may optionally include that the ultrasound scanner is implemented to communicate the audio to an operator of the ultrasound system while the at least one transducer array transmits the ultrasound and receives the reflections.

Example 8 is the ultrasound system of example 3 that may optionally include that the processor system is implemented to generate, based on at least one of the reflections of the ultrasound, the infrared radiation, the audio, the image, and the inference, guidance instructions, wherein the ultrasound scanner is implemented to indicate to an operator of the ultrasound scanner an orientation of the ultrasound scanner based on the guidance instructions.

Example 9 is the ultrasound system of example 8 that may optionally include that the ultrasound scanner is implemented to indicate the orientation such that the indication is imperceptible to the patient.

Example 10 is the ultrasound system of example 8 that may optionally include that the indication includes to adjust a position of a weight of the inside the ultrasound scanner.

Example 11 is the ultrasound system of example 10 that may optionally include that the weight includes a battery implemented to provide power to the ultrasound scanner.

Example 12 is the ultrasound system of example 8 that may optionally include that the ultrasound scanner includes an additional display device implemented to display a visual representation of the indication of the orientation.

Example 13 is the ultrasound system of example 3 that may optionally include that the at least one transducer array includes a piezoelectric micro-machined ultrasonic transducer (PMUT) array, and the PMUT array includes the microphone.

Example 14 is the ultrasound system of example 3 that may optionally include that the microphone includes a vibrating element to detect the audio, the vibrating element including a region of an enclosure of the ultrasound scanner.

Example 15 is the ultrasound system of example 2 that may optionally include an ultrasound machine having a speaker, wherein the ultrasound scanner includes a microphone and the ultrasound machine is implemented to broadcast audio including operation instructions from the speaker to the microphone of the ultrasound scanner, and the at least one transducer array is implemented to transmit the ultrasound and receive the reflections based on the operation instructions.

Example 16 is the ultrasound system of example 15 that may optionally include that the audio occupies a bandwidth outside a human-perceptible bandwidth.

Example 17 is the ultrasound system of example 2 that may optionally include that the ultrasound scanner includes a microphone implemented to detect audio caused by tactile gestures on the ultrasound scanner, wherein the processor system is implemented to edit a medical worksheet based on the audio caused by the tactile gestures.

Example 18 is the ultrasound system of example 2 that may optionally include that the ultrasound scanner includes a microphone implemented to detect audio caused by tactile gestures on the ultrasound scanner, wherein the processor system is implemented to configure, based on the audio caused by the tactile gestures, the at least one transducer array to transmit the ultrasound and receive the reflections.

Example 19 is an ultrasound system including a display device configured to display a user interface for the ultrasound system, an ultrasound scanner having at least one transducer array configured to transmit ultrasound at a patient anatomy and receive reflections of the ultrasound from the patient anatomy, and an infrared sensor configured to receive infrared radiation from the patient anatomy. The ultrasound system also includes a processor system configured to: generate, based on the reflections of the ultrasound, an ultrasound image of the patient anatomy; generate, based on the infrared radiation, an infrared image of the patient anatomy; and cause the user interface to display the ultrasound image and the infrared image.

Example 20 is the ultrasound system of example 19 that may optionally include that the processor system is implemented to cause the user interface to display the infrared image prior to the display of the ultrasound image.

Example 21 is the ultrasound system of example 19 that may optionally include that the processor system is implemented to cause the user interface to display the infrared image and the ultrasound image simultaneously.

Example 22 is the ultrasound system of example 19 that may optionally include that the processor system is implemented to generate, based on the ultrasound image and the infrared image, a combination image, and cause the user interface to display the combination image.

Example 23 is the ultrasound system of example 22 that may optionally include that the combination image includes a three-dimensional image of the patient anatomy.

Example 24 is the ultrasound system of example 19 that may optionally include that the processor system is implemented to: detect, based on at least one of the ultrasound image and the infrared image, an interventional instrument; determine a position of the interventional instrument relative to the patient anatomy; and cause, based on the position of the interventional instrument, the user interface to cease the display of the infrared image and start the display of the ultrasound image.

Example 25 is the ultrasound system of example 24 that may optionally include that the interventional instrument has a heat pattern applied to it prior to insertion into the patient, and the processor system is implemented to detect the interventional instrument in the infrared image and determine the position based on the heat pattern.

Example 26 is the ultrasound system of example 19 that may optionally include that the infrared image includes an interventional instrument having a first temperature that is different from a second temperature of the patient anatomy.

Example 27 is the ultrasound system of example 26 that may optionally include a temperature adjustment device implemented to set the interventional instrument to the first temperature prior to insertion of the interventional instrument into the patient.

Example 28 is the ultrasound system of example 27 that may optionally include that the temperature adjustment device is implemented to maintain the interventional instrument at the first temperature during the insertion.

Example 29 is the ultrasound system of example 19 that may optionally include that the ultrasound scanner includes a microphone implemented to receive audio from the patient anatomy, and the processor system is implemented to generate an inference of the patient anatomy based on the audio and the ultrasound image.

Example 30 is the ultrasound system of example 29 that may optionally include that the processor system is implemented to generate a visual representation of the audio and cause the user interface to display the visual representation.

Example 31 is the ultrasound system of example 29 that may optionally include that the processor system is implemented to classify the patient anatomy based on the audio.

Example 32 is the ultrasound system of example 19 that may optionally include that the ultrasound scanner includes a microphone implemented to receive audio from the patient anatomy, and the processor system is implemented to: generate, based on the reflections of the ultrasound, additional audio; combine the audio and the additional audio to form combined audio; and communicate the combined audio to an operator of the ultrasound system.

Example 33 is the ultrasound system of example 19 that may optionally include that the processor system is implemented to generate, based on the ultrasound image, a label for the patient anatomy, and cause the label to be applied to the patient anatomy in the infrared image.

Example 34 is an ultrasound system including an ultrasound scanner having at least one transducer array configured to transmit ultrasound at a patient anatomy and receive reflections of the ultrasound from the patient anatomy; and at least one sensor different from the at least one transducer array and configured to generate sensor data based on radiated energy from the patient anatomy. The ultrasound system also has a processor system configured to generate, based on the reflections of the ultrasound and the sensor data, an image of the patient anatomy.

Example 35 is the ultrasound system of example 34 that may optionally include that the at least one sensor includes an infrared sensor, the radiated energy includes infrared radiation, and the sensor data includes heat data.

Example 36 is the ultrasound system of example 34 that may optionally include that the at least one sensor includes a microphone, the radiated energy includes sound radiation, and the sensor data includes audio data.

Example 37 is the ultrasound system of example 34 that may optionally include that the at least one sensor includes an infrared sensor and a microphone, the radiated energy includes infrared radiation and sound radiation, and the sensor data includes heat data and audio data.

Example 38 is the ultrasound system of example 34 that may optionally include that the at least one sensor includes a magnetic sensor, the radiated energy includes magnetic radiation, and the sensor data includes magnetic field data.

Example 39 is the ultrasound system of example 34 that may optionally include that the ultrasound scanner is a wearable ultrasound scanner implemented to be patient worn, and the processor system is implemented to communicate the image of the patient anatomy over a network to a medical archiver.

Example 40 is the ultrasound system of example 34 that may optionally include that the ultrasound scanner is a wearable ultrasound scanner implemented to be patient worn, and the processor system is implemented to communicate the image of the patient anatomy over a network to a clinician.

Example 41 is a method including the operations performed by the ultrasound systems of one or more of examples 1-40.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. An ultrasound system comprising:
a display device configured to display a user interface for the ultrasound system;
an ultrasound scanner having at least one transducer array configured to transmit ultrasound at a patient anatomy and receive reflections of the ultrasound from the patient anatomy, wherein the ultrasound scanner includes an infrared sensor configured to receive infrared radiation emitted by the patient anatomy and generate heat data based on the infrared radiation;
and a processor system configured to:
generate, based on the reflections of the ultrasound and the heat data, an image of the patient anatomy; and
cause the user interface to display the image of the patient anatomy.

2. (canceled)

3. The ultrasound system as described in claim 1, wherein the ultrasound scanner includes a microphone implemented to receive audio from the patient anatomy, and the processor system is configured to implement a machine-learned model that generates, based on the reflections of the ultrasound and the audio, an inference for the patient anatomy.

4. The ultrasound system as described in claim 3, wherein the inference includes at least one of a label, a classification, a segmentation, and an additional image.

5. The ultrasound system as described in claim 3, wherein the processor system is implemented to generate, based on at least one of the reflections of the ultrasound, the infrared radiation, the audio, the image, and the inference, guidance instructions, wherein the ultrasound scanner is implemented to indicate to an operator of the ultrasound scanner an orientation of the ultrasound scanner based on the guidance instructions.

6. The ultrasound system as described in claim 5, wherein the indication includes to adjust a position of a weight of the inside the ultrasound scanner.

7. The ultrasound system as described in claim 5, wherein the ultrasound scanner includes an additional display device implemented to display a visual representation of the indication of the orientation.

8. The ultrasound system as described in claim 12, further comprising an ultrasound machine having a speaker, wherein the ultrasound scanner includes a microphone and the ultrasound machine is implemented to broadcast audio including operation instructions from the speaker to the microphone of the ultrasound scanner, and the at least one transducer array is implemented to transmit the ultrasound and receive the reflections based on the operation instructions.

9. The ultrasound system as described in claim 12, wherein the ultrasound scanner includes a microphone implemented to detect audio caused by tactile gestures on the ultrasound scanner.

10. An ultrasound system comprising:
a display device configured to display a user interface for the ultrasound system;
an ultrasound scanner having at least one transducer array configured to transmit ultrasound at a patient anatomy and receive reflections of the ultrasound from the patient anatomy, wherein the ultrasound scanner includes an infrared sensor configured to receive infrared radiation emitted by the patient anatomy;
and a processor system configured to:
generate, based on the reflections of the ultrasound, an ultrasound image of the patient anatomy;
generate, based on the infrared radiation, an infrared image of the patient anatomy; and
cause the user interface to display the ultrasound image and the infrared image.

11. The ultrasound system as described in claim 10, wherein the processor system is implemented to cause the user interface to display the infrared image prior to the display of the ultrasound image.

12. The ultrasound system as described in claim 10, wherein the processor system is implemented to cause the user interface to display the infrared image and the ultrasound image simultaneously.

13. The ultrasound system as described in claim 10, wherein the processor system is implemented to generate, based on the ultrasound image and the infrared image, a combination image, and cause the user interface to display the combination image.

14. The ultrasound system as described in claim 10, wherein the processor system is implemented to:
detect, based on at least one of the ultrasound image and the infrared image, an interventional instrument;
determine a position of the interventional instrument relative to the patient anatomy; and
cause, based on the position of the interventional instrument, the user interface to cease the display of the infrared image and start the display of the ultrasound image.

15. The ultrasound system as described in claim 14, wherein the interventional instrument has a heat pattern applied to it prior to insertion into the patient, and the processor system is implemented to detect the interventional instrument in the infrared image and determine the position based on the heat pattern.

16. The ultrasound system as described in claim 10, wherein the infrared image includes an interventional instrument having a first temperature that is different from a second temperature of the patient anatomy.

17. An ultrasound system comprising:
an ultrasound scanner including:
at least one transducer array configured to transmit ultrasound at a patient anatomy and receive reflections of the ultrasound from the patient anatomy; and
at least one sensor different from the at least one transducer array and configured to generate sensor data based on energy emitted by the patient anatomy; and
a processor system configured to generate, based on the reflections of the ultrasound and the sensor data, an image of the patient anatomy.

18. The ultrasound system as described in claim 17, wherein the at least one sensor includes an infrared sensor, the energy emitted by the patient anatomy includes infrared radiation, and the sensor data includes heat data.

19. The ultrasound system as described in claim 17, wherein the at least one sensor includes a microphone, the energy emitted by the patient anatomy includes sound radiation, and the sensor data includes audio data.

20. The ultrasound system as described in claim 17, wherein the ultrasound scanner is a wearable ultrasound scanner implemented to be patient worn, and the processor system is implemented to communicate the image of the patient anatomy over a network to a medical archiver or a clinician.
